SEQUENCE LISTING (178 sequences)

SEQ ID NO: 1 - RNA, 19 nt, Homo sapiens
gaagaaagcccucagaauc

SEQ ID NO: 2 - RNA, 19 nt, Homo sapiens
gugccgagcugcaugacua

SEQ ID NO: 3 - RNA, 19 nt, Homo sapiens
gcacagcagucaucguggu

SEQ ID NO: 4 - RNA, 19 nt, Homo sapiens
ucgcugaccgugaacgaua

SEQ ID NO: 5 - RNA, 19 nt, Homo sapiens
cgacuuuacccuucgacua

SEQ ID NO: 6 - RNA, 19 nt, Homo sapiens
cuacagagauggcuucaac

SEQ ID NO: 7 - RNA, 19 nt, Homo sapiens
caacucccuugacagugua

SEQ ID NO: 8 - RNA, 19 nt, Homo sapiens
ugaggugacuccaguaucu

SEQ ID NO: 9 - RNA, 19 nt, Homo sapiens
aaagggaugugaauuauug

SEQ ID NO: 10 - RNA, 19 nt, Homo sapiens
gaaaacaucuauaccauug

SEQ ID NO: 11 - RNA, 19 nt, Homo sapiens
gagcggaggucggucagaa

SEQ ID NO: 12 - RNA, 19 nt, Homo sapiens
cagcaacccucacaaccuu

SEQ ID NO: 13 - RNA, 19 nt, Homo sapiens
gggcgugacuuccacauga

SEQ ID NO: 14 - RNA, 19 nt, Homo sapiens
cggagagcuucgugcuaaa

SEQ ID NO: 15 - RNA, 19 nt, Homo sapiens
acaauaggagccaggcgca

SEQ ID NO: 16 - RNA, 19 nt, Homo sapiens
gcaaugacagcggcaccua

SEQ ID NO: 17 - RNA, 19 nt, Homo sapiens
gaacccagauuuauguaau

SEQ ID NO: 18 - RNA, 19 nt, Homo sapiens
gaaccucacuauccaagga

SEQ ID NO: 19 - RNA, 19 nt, Homo sapiens
auauaaaguuggaugcgga

SEQ ID NO: 20 - RNA, 19 nt, Homo sapiens
cuaggaagcuccaguucga

SEQ ID NO: 21 - RNA, 19 nt, Homo sapiens
gaaccuggcugcuguauug

SEQ ID NO: 22 - RNA, 19 nt, Homo sapiens
ggaagucacugccauggaa

SEQ ID NO: 23 - RNA, 19 nt, Homo sapiens
ugaaaucacugcauuacaa

SEQ ID NO: 24 - RNA, 19 nt, Homo sapiens
ggacuuuauuugccauaua

SEQ ID NO: 25 - DNA, 19 bp, Artificial Sequence
  Description of Artificial Sequence Synthetic oligonucleotide
tgctgtgtgtggggacaac

SEQ ID NO: 26 - DNA, 19 bp, Artificial Sequence
  Description of Artificial Sequence Synthetic oligonucleotide
gggctgcaagggcttcttc

SEQ ID NO: 27 - DNA, 19 bp, Artificial Sequence
  Description of Artificial Sequence Synthetic oligonucleotide
gcgcacagtgcagaaaaac

SEQ ID NO: 28 - DNA, 19 bp, Artificial Sequence
  Description of Artificial Sequence Synthetic oligonucleotide
cctcacgcagagctccatc

SEQ ID NO: 29 - DNA, 19 bp, Artificial Sequence
  Description of Artificial Sequence Synthetic oligonucleotide
catggtgtacttcaacttc

SEQ ID NO: 30 - DNA, 19 bp, Artificial Sequence
  Description of Artificial Sequence Synthetic oligonucleotide
gtgtacttcaacttctttg

SEQ ID NO: 31 - DNA, 19 bp, Artificial Sequence
  Description of Artificial Sequence Synthetic oligonucleotide
gcaccttcatggacggcta

SEQ ID NO: 32 - DNA, 19 bp, Artificial Sequence
  Description of Artificial Sequence Synthetic oligonucleotide
gcattatggtgtccgcaca

SEQ ID NO: 33 - DNA, 19 bp, Artificial Sequence
  Description of Artificial Sequence Synthetic oligonucleotide
tgaaggaagttgtccgaac

SEQ ID NO: 34 - DNA, 19 bp, Artificial Sequence
  Description of Artificial Sequence Synthetic oligonucleotide
ctgcagaaccgcatcgcca

SEQ ID NO: 35 - DNA, 19 bp, Artificial Sequence
  Description of Artificial Sequence Synthetic oligonucleotide
ccacgtgactttcaacaat

SEQ ID NO: 36 - DNA, 19 bp, Artificial Sequence
  Description of Artificial Sequence Synthetic oligonucleotide
acattcagatgcacaacta

SEQ ID NO: 37 - DNA, 19 bp, Artificial Sequence
  Description of Artificial Sequence Synthetic oligonucleotide
ggacaagcgtcgccggaat

SEQ ID NO: 38 - DNA, 19 bp, Artificial Sequence
  Description of Artificial Sequence Synthetic oligonucleotide
ccaccttgcttgtaccaaa

SEQ ID NO: 39 - DNA, 19 bp, Artificial Sequence
  Description of Artificial Sequence Synthetic oligonucleotide
ggagaagcatcgtgagtga

SEQ ID NO: 40 - DNA, 19 bp, Artificial Sequence
  Description of Artificial Sequence Synthetic oligonucleotide
ccactttggtgtttcataa

SEQ ID NO: 41 - DNA, 19 bp, Artificial Sequence
  Description of Artificial Sequence Synthetic oligonucleotide
tcaggttgctggtcaaatt

SEQ ID NO: 42 - DNA, 19 bp, Artificial Sequence
  Description of Artificial Sequence Synthetic oligonucleotide
cgttatataggaaccgtaa

SEQ ID NO: 43 - DNA, 19 bp, Artificial Sequence
  Description of Artificial Sequence Synthetic oligonucleotide
ggataactcggccatgaga

SEQ ID NO: 44 - DNA, 19 bp, Artificial Sequence
  Description of Artificial Sequence Synthetic oligonucleotide
caagaagagttacctcagc

SEQ ID NO: 45 - DNA, 19 bp, Artificial Sequence
  Description of Artificial Sequence Synthetic oligonucleotide
gaactcacctcctgattat

SEQ ID NO: 46 - DNA, 19 bp, Artificial Sequence
  Description of Artificial Sequence Synthetic oligonucleotide
gcttagtaatgctacgtgt

SEQ ID NO: 47 - DNA, 19 bp, Artificial Sequence
  Description of Artificial Sequence Synthetic oligonucleotide
gaccatacctcataacaag

SEQ ID NO: 48 - DNA, 19 bp, Artificial Sequence
  Description of Artificial Sequence Synthetic oligonucleotide
tgaaagacctccaccaatc

SEQ ID NO: 49 - DNA, 19 bp, Artificial Sequence
  Description of Artificial Sequence Synthetic oligonucleotide
gatgaaggctccaggtgtt

SEQ ID NO: 50 - DNA, 19 bp, Artificial Sequence
  Description of Artificial Sequence Synthetic oligonucleotide
tatcagcatttacgactta

SEQ ID NO: 51 - DNA, 19 bp, Artificial Sequence
  Description of Artificial Sequence Synthetic oligonucleotide
ccacatggccagctgacta

SEQ ID NO: 52 - DNA, 19 bp, Artificial Sequence
  Description of Artificial Sequence Synthetic oligonucleotide
caacccgactatcagacta

SEQ ID NO: 53 - DNA, 19 bp, Artificial Sequence
  Description of Artificial Sequence Synthetic oligonucleotide
gaatgaatcctcacctaac

SEQ ID NO: 54 - DNA, 19 bp, Artificial Sequence
  Description of Artificial Sequence Synthetic oligonucleotide
gcaacaagtttgacggtga

SEQ ID NO: 55 - DNA, 19 bp, Artificial Sequence
  Description of Artificial Sequence Synthetic oligonucleotide
ggaagtgcatttcaagatc

SEQ ID NO: 56 - DNA, 19 bp, Artificial Sequence
  Description of Artificial Sequence Synthetic oligonucleotide
cgagaacaacgaagactat

SEQ ID NO: 57 - DNA, 19 bp, Artificial Sequence
  Description of Artificial Sequence Synthetic oligonucleotide
caagagcactcaggcaaac

SEQ ID NO: 58 - DNA, 19 bp, Artificial Sequence
  Description of Artificial Sequence Synthetic oligonucleotide
aaagagaccttcagccgac

SEQ ID NO: 59 - DNA, 21 bp, Artificial Sequence
  Description of Artificial Sequence Synthetic oligonucleotide
aaggaggtttgtataagtaat

SEQ ID NO: 60 - DNA, 21 bp, Artificial Sequence
  Description of Artificial Sequence Synthetic oligonucleotide
caggagcgcattgcacatcaa

SEQ ID NO: 61 - DNA, 21 bp, Artificial Sequence
  Description of Artificial Sequence Synthetic oligonucleotide
ttcagtgggaattgcagcata

SEQ ID NO: 62 - DNA, 21 bp, Artificial Sequence
  Description of Artificial Sequence Synthetic oligonucleotide
ctgatgagtcttgatctagat

SEQ ID NO: 63 - RNA, 19 nt, Artificial Sequence
  Description of Artificial Sequence Synthetic oligonucleotide
gaacgucaccaacuacuuu

SEQ ID NO: 64 - RNA, 19 nt, Artificial Sequence
  Description of Artificial Sequence Synthetic oligonucleotide
caugcugggugucuauuug

SEQ ID NO: 65 - RNA, 19 nt, Artificial Sequence
  Description of Artificial Sequence Synthetic oligonucleotide
caacugcggucagccaaag

SEQ ID NO: 66 - RNA, 19 nt, Artificial Sequence
  Description of Artificial Sequence Synthetic oligonucleotide
ccaaguggccugucucuuu

SEQ ID NO: 67 - RNA, 19 nt, Artificial Sequence
  Description of Artificial Sequence Synthetic oligonucleotide
ugagcuacaugguauauuu

SEQ ID NO: 68 - RNA, 19 nt, Artificial Sequence
  Description of Artificial Sequence Synthetic oligonucleotide
gggauggaaccacgaauga

SEQ ID NO: 69 - RNA, 19 nt, Artificial Sequence
  Description of Artificial Sequence Synthetic oligonucleotide
gauggaaccacgaaugaaa

SEQ ID NO: 70 - RNA, 19 nt, Artificial Sequence
  Description of Artificial Sequence Synthetic oligonucleotide
gaaccgagacuuccgcuac

SEQ ID NO: 71 - RNA, 19 nt, Artificial Sequence
  Description of Artificial Sequence Synthetic oligonucleotide
gaaacaggauucaguguga

SEQ ID NO: 72 - RNA, 19 nt, Artificial Sequence
  Description of Artificial Sequence Synthetic oligonucleotide
acaaaggaguuacaucuua

SEQ ID NO: 73 - RNA, 19 nt, Artificial Sequence
  Description of Artificial Sequence Synthetic oligonucleotide
aaagggagauucuaguaua

SEQ ID NO: 74 - RNA, 19 nt, Artificial Sequence
  Description of Artificial Sequence Synthetic oligonucleotide
aaacagaaaucgaaacaga

SEQ ID NO: 75 - RNA, 19 nt, Artificial Sequence
  Description of Artificial Sequence Synthetic oligonucleotide
ccguauggcucaacuucga

SEQ ID NO: 76 - RNA, 19 nt, Artificial Sequence
  Description of Artificial Sequence Synthetic oligonucleotide
aggcaggcgucgaagagua

SEQ ID NO: 77 - RNA, 19 nt, Artificial Sequence
  Description of Artificial Sequence Synthetic oligonucleotide
gcucuacgaagaucuggaa

SEQ ID NO: 78 - RNA, 19 nt, Artificial Sequence
  Description of Artificial Sequence Synthetic oligonucleotide
ggagcgcauugcacaucaa

SEQ ID NO: 79 - RNA, 19 nt, Artificial Sequence
  Description of Artificial Sequence Synthetic oligonucleotide
guacagcgcccacgcauuc

SEQ ID NO: 80 - RNA, 19 nt, Artificial Sequence
  Description of Artificial Sequence Synthetic oligonucleotide
gaaacagaacgcggcucua

SEQ ID NO: 81 - RNA, 19 nt, Artificial Sequence
  Description of Artificial Sequence Synthetic oligonucleotide
gaacgcggcucuacgcaag

SEQ ID NO: 82 - RNA, 19 nt, Artificial Sequence
  Description of Artificial Sequence Synthetic oligonucleotide
agaguucagaggagggaga

SEQ ID NO: 83 - RNA, 41 nt, Artificial Sequence
  Description of Artificial Sequence Synthetic oligonucleotide
ggcuggggugcgacauuauguucguuaaggaucaaucgcuc

SEQ ID NO: 84 - RNA, 40 nt, Artificial Sequence
  Description of Artificial Sequence Synthetic oligonucleotide
uuaugaugcaaaaacgaacuggaauggccaugcagguaca

SEQ ID NO: 85 - RNA, 41 nt, Artificial Sequence
  Description of Artificial Sequence Synthetic oligonucleotide
gguuggggugcgacauuauguucguuaaggaucaaucgcuc

SEQ ID NO: 86 - RNA, 41 nt, Artificial Sequence
  Description of Artificial Sequence Synthetic oligonucleotide
gauuuggagagcauuauguuagguuaaggaucaaucuucua

SEQ ID NO: 87 - RNA, 41 nt, Artificial Sequence
  Description of Artificial Sequence Synthetic oligonucleotide
ggcuggggugcgacauuauguucguuaaggaucaaucgcuu

SEQ ID NO: 88 - RNA, 83 nt, Artificial Sequence
  Description of Artificial Sequence Synthetic oligonucleotide
gggguaaacgaacaggauuuuuuuaacgagcuauauuguuuccuguugcccguccgucuagucgugaagagagcaagguuacu

SEQ ID NO: 89 - RNA, 83 nt, Artificial Sequence
  Description of Artificial Sequence Synthetic oligonucleotide
gggguaaacgaacaggacgacgggucgaagcugaauagguaaccaaucacggcauaacuagucgugaagagagcaagguuacu

SEQ ID NO: 90 - RNA, 83 nt, Artificial Sequence
  Description of Artificial Sequence Synthetic oligonucleotide
gggguaaacgaacaggaugagggagcaaaaagggcgaaaaugcaguaacuaaacguucuagucgugaagagagcaagguuacu

SEQ ID NO: 91 - RNA, 83 nt, Artificial Sequence
  Description of Artificial Sequence Synthetic oligonucleotide
gggguaaacgaacaggauuuuuuuaacgagcuauauuauuuccuguugcccguccgucuagucgugaagagagcaagguuacu

SEQ ID NO: 92 - RNA, 84 nt, Artificial Sequence
  Description of Artificial Sequence Synthetic oligonucleotide
gggguaaacgaacaggaaccauuaaaucauaaggagaaagaugaugugcgcgacauaacuagucgugaagagagcaagguuacu

SEQ ID NO: 93 - RNA, 89 nt, Artificial Sequence
  Description of Artificial Sequence Synthetic oligonucleotide
gggagacaaacaaagagcgacaagggcagcggguuaucacguugggaacgggccaucaacucuucucaccacccagaagaagccagaag

SEQ ID NO: 94 - RNA, 89 nt, Artificial Sequence
  Description of Artificial Sequence Synthetic oligonucleotide
gggagacaaacaaagagcgacaagggcaguagugagggauucaccagagugaaugcgcuccucggaaaucacccagaagaagccagaag

SEQ ID NO: 95 - RNA, 89 nt, Artificial Sequence
  Description of Artificial Sequence Synthetic oligonucleotide
gggagacaaacaaagagcgacaagggcagaacgggcaauguccaaggugaggcaguuuguauggacacacacccagaagaagccagaag

SEQ ID NO: 96 - RNA, 89 nt, Artificial Sequence
  Description of Artificial Sequence Synthetic oligonucleotide
gggagacaaacaaagagcgacaagggcaguugagauugaggagucagaccugcgucucuaguaacaaugcacccagaagaagccagaag

SEQ ID NO: 97 - RNA, 89 nt, Artificial Sequence
  Description of Artificial Sequence Synthetic oligonucleotide
gggagacaaacaaagagcgacaagggcagaguggacggucggcuagagccgggaggaauuccuugugaccacccagaagaagccagaag

SEQ ID NO: 98 - RNA, 89 nt, Artificial Sequence
  Description of Artificial Sequence Synthetic oligonucleotide
gggagacaaacaaagagcgacaagggcaguuugacaauguaccuuuaauuacggauuguaccuugggcgcacccagaagaagccagaag

SEQ ID NO: 99 - RNA, 35 nt, Artificial Sequence
  Description of Artificial Sequence Synthetic oligonucleotide
gggagagaggaagagggaugggccgacgugccgca

SEQ ID NO: 100 - RNA, 89 nt, Artificial Sequence
  Description of Artificial Sequence Synthetic oligonucleotide
gggagacaaacaaagagcgacaagggcagaauuacaauagcuauaguccgggcaccaugcuuguaaauccacccagaagaagccagaag

SEQ ID NO: 101 - RNA, 89 nt, Artificial Sequence
  Description of Artificial Sequence Synthetic oligonucleotide
gggagacaaacaaagagcgacaagggcagguaggacgcuagcagacuagaauguaucuaugcuuagauccacccagaagaagccagaag

SEQ ID NO: 102 - RNA, 89 nt, Artificial Sequence
  Description of Artificial Sequence Synthetic oligonucleotide
gggagacaaacaaagagcgacaagggcaggcuaguauuacaaugucguggaaaagccgugcgggguauccacccagaagaagccagaag

SEQ ID NO: 103 - RNA, 89 nt, Artificial Sequence
  Description of Artificial Sequence Synthetic oligonucleotide
gggagacaaacaaagagcgacaagggcagggagccauucuugaaauugucaguuugauugugcucaggucacccagaagaagccagaag

SEQ ID NO: 104 - RNA, 89 nt, Artificial Sequence
  Description of Artificial Sequence Synthetic oligonucleotide
gggagacaaacaaagagcgacaagggcagaaaguacaaugguugacauauaccgucgguuuauccuaugcacccagaagaagccagaag

SEQ ID NO: 105 - RNA, 89 nt, Artificial Sequence
  Description of Artificial Sequence Synthetic oligonucleotide
gggagacaaacaaagagcgacaagggcagguaggcuaucgcugcuugaucgucugaucagagccuauaccacccagaagaagccagaag

SEQ ID NO: 106 - RNA, 83 nt, Artificial Sequence
  Description of Artificial Sequence Synthetic oligonucleotide
gggguaaacgaacaggaaacagauggccaacacaggcgaagcauagacuaggaacggcuagucgugaagagagcaagguuacu

SEQ ID NO: 107 - RNA, 81 nt, Artificial Sequence
  Description of Artificial Sequence Synthetic oligonucleotide
gggguaaacgaacaggacuugaugugaaaaggcgacgcgaugagacgaagggcuucuagucgugaagagagcaagguuacu

SEQ ID NO: 108 - RNA, 83 nt, Artificial Sequence
  Description of Artificial Sequence Synthetic oligonucleotide
gggguaaacgaacaggaaguagacuagacggcggcgauaaccagauaacgacauucucuagucgugaagagagcaagguuacu

SEQ ID NO: 109 - RNA, 83 nt, Artificial Sequence
  Description of Artificial Sequence Synthetic oligonucleotide
gggguaaacgaacaggaccgagugagacggguaguggacaaaugaaguagugugguccuagucgugaagagagcaagguuacu

SEQ ID NO: 110 - RNA, 83 nt, Artificial Sequence
  Description of Artificial Sequence Synthetic oligonucleotide
gggguaaacgaacaggacuuuuaauuucacgccgcacgauccggaaaaacgacuugacuagucgugaagagagcaagguuacu

SEQ ID NO: 111 - DNA, 21 bp, Unknown
  Description of Unknown target sequence
ctgattaatatatttaatata

SEQ ID NO: 112 - DNA, 21 bp, Unknown
  Description of Unknown target sequence
ctccttccacatgtacataaa

SEQ ID NO: 113 - DNA, 21 bp, Unknown
  Description of Unknown target sequence
cagcattatggtgtccgcaca

SEQ ID NO: 114 - DNA, 21 bp, Unknown
  Description of Unknown target sequence
cagcaccttcatggacggcta

SEQ ID NO: 115 - DNA, 19 bp, Unknown
  Description of Unknown target sequence
gcaccttcatggacggcta

SEQ ID NO: 116 - DNA, 18 bp, Unknown
  Description of Unknown target sequence
gcatatggtgtccgcaca

SEQ ID NO: 117 - DNA, 18 bp, Unknown
  Description of Unknown target sequence
tgaaggaagtgtccgaac

SEQ ID NO: 118 - DNA, 19 bp, Unknown
  Description of Unknown target sequence
ctgcagaaccgcatcgcca

SEQ ID NO: 119 - DNA, 19 bp, Unknown
  Description of Unknown target sequence
tgctgtgtgtggggacaac

SEQ ID NO: 120 - DNA, 19 bp, Unknown
  Description of Unknown target sequence
gggctgcaagggcttcttc

SEQ ID NO: 121 - DNA, 19 bp, Unknown
  Description of Unknown target sequence
gcgcacagtgcagaaaaac

SEQ ID NO: 122 - DNA, 17 bp, Unknown
  Description of Unknown target sequence
cagtggctctgactact

SEQ ID NO: 123 - DNA, 19 bp, Unknown
  Description of Unknown target sequence
ccacttctccacaccttga

SEQ ID NO: 124 - DNA, 18 bp, Unknown
  Description of Unknown target sequence
ggcttgagctgcagaatg

SEQ ID NO: 125 - DNA, 21 bp, Unknown
  Description of Unknown target sequence
cacagcttgcttgtcgatgtc

SEQ ID NO: 126 - DNA, 25 bp, Unknown
  Description of Unknown target sequence
ggtccctgcacagcttgcttgtcga

SEQ ID NO: 127 - DNA, 25 bp, Unknown
  Description of Unknown target sequence
ccggttctctggaggtcatccgcaa

SEQ ID NO: 128 - DNA, 25 bp, Unknown
  Description of Unknown target sequence
cagcattatggtgtccgcacatgtg

SEQ ID NO: 129 - DNA, 21 bp, Unknown
  Description of Unknown target sequence
aatgttgacggacagcctatt

SEQ ID NO: 130 - DNA, 21 bp, Unknown
  Description of Unknown target sequence
aagagtacggccctgaagaag

SEQ ID NO: 131 - DNA, 21 bp, Unknown
  Description of Unknown target sequence
aaggaggtttgtataagtaat

SEQ ID NO: 132 - DNA, 21 bp, Unknown
  Description of Unknown target sequence
caggagcgcattgcacatcaa

SEQ ID NO: 133 - DNA, 21 bp, Unknown
  Description of Unknown target sequence
ccctattagatacacttctta

SEQ ID NO: 134 - DNA, 21 bp, Unknown
  Description of Unknown target sequence
taaggaggtttgtataagtaa

SEQ ID NO: 135 - DNA, 21 bp, Unknown
  Description of Unknown target sequence
cctagtcaagcctgagaatta

SEQ ID NO: 136 - DNA, 19 bp, Unknown
  Description of Unknown target sequence
ctgccagtgtatactctga

SEQ ID NO: 137 - DNA, 19 bp, Unknown
  Description of Unknown target sequence
atacactcggtagctgtgg

SEQ ID NO: 138 - RNA, 70 nt, Artificial Sequence
  Description of Artificial Sequence Synthetic oligonucleotide
gggagagagauauaagggccuccugauacccgcugcuaucuggaccgaucccauuaccaaauucucuccc

SEQ ID NO: 139 - RNA, 83 nt, Artificial Sequence
  Description of Artificial Sequence Synthetic oligonucleotide
gggguaaacgaacaggaagacggcgcaauaagacagacuaggacacgauuagagguacuagucgugaagagagcaagguuacu

SEQ ID NO: 140 - RNA, 83 nt, Artificial Sequence
  Description of Artificial Sequence Synthetic oligonucleotide
gggguaaacgaacaggauaaaagaaaacaacuagcgcgacgagagaauaaaaugaaacuagucgugaagagagcaagguuacu

SEQ ID NO: 141 - RNA, 83 nt, Artificial Sequence
  Description of Artificial Sequence Synthetic oligonucleotide
gggguaaacgaacaggauaauuguuggggaaauaaauugcugggaacgacuuaaaagcuagucgugaagagagcaagguuacu

SEQ ID NO: 142 - RNA, 83 nt, Artificial Sequence
  Description of Artificial Sequence Synthetic oligonucleotide
gggguaaacgaacaggaguuaaucaugagguagguaacaaaaggcaacggccaauaacuagucgugaagagagcaagguuacu

SEQ ID NO: 143 - RNA, 83 nt, Artificial Sequence
  Description of Artificial Sequence Synthetic oligonucleotide
gggguaaacgaacaggauaaccaugcaaauaacaagcaaacagagaacucacgccagcuagucgugaagagagcaagguuacu

SEQ ID NO: 144 - RNA, 83 nt, Artificial Sequence
  Description of Artificial Sequence Synthetic oligonucleotide
gggguaaacgaacaggaagggagucgauuugaguuguaauuugaccuauguuauaaucuagucgugaagagagcaagguuacu

SEQ ID NO: 145 - RNA, 83 nt, Artificial Sequence
  Description of Artificial Sequence Synthetic oligonucleotide
gggguaaacgaacaggaauggcuacaguaucgaugcaguuuucgaaugaaguagaaacuagucgugaagagagcaagguuacu

SEQ ID NO: 146 - RNA, 83 nt, Artificial Sequence
  Description of Artificial Sequence Synthetic oligonucleotide
gggguaaacgaacaggacaggacagcaagcaguagaaaacaagccacgaaggggacucuagucgugaagagagcaagguuacu

SEQ ID NO: 147 - RNA, 83 nt, Artificial Sequence
  Description of Artificial Sequence Synthetic oligonucleotide
gggguaaacgaacaggauuuuggacugucuagccgauguacuuaaguuuaucauuuucuagucgugaagagagcaagguuacu

SEQ ID NO: 148 - RNA, 83 nt, Artificial Sequence
  Description of Artificial Sequence Synthetic oligonucleotide
gggguaaacgaacaggagcagucgcuggcuucauuuuuuuuuuuuuuuugugcucaacuagucgugaagagagcaagguuacu

SEQ ID NO: 149 - RNA, 51 nt, Artificial Sequence
  Description of Artificial Sequence Synthetic oligonucleotide
aggagacacaugugacaagaggcuaugauccugaaugcauccuuggcaaac

SEQ ID NO: 150 - RNA, 51 nt, Artificial Sequence
  Description of Artificial Sequence Synthetic oligonucleotide
aggagaaucaugaguuaucuguguaaggaaccaaagccaugcuuaucaaac

SEQ ID NO: 151 - DNA, 120 bp, Homo sapiens
acgtgtgccgtgtgcggggacaacgccgcctgccagcactacggcgtgcgaacctgcgagggctgcaagggctttttcaagagaacagtgcagaaaaatgcaaaatatgtttgcctggca

SEQ ID NO: 152 - DNA, 120 bp, Homo sapiens
cgctgtgctgtgtgtggggacaacgcttcatgccagcattatggtgtccgcacatgtgagggctgcaagggcttcttcaagcgcacagtgcagaaaaacgccaagtacatctgcctggct

SEQ ID NO: 153 - DNA, 120 bp, Homo sapiens
ctgtgcgctgtgtgtggggacaacgcggcctgccaacactacggcgtgcgcacctgtgagggctgcaaaggcttctttaagcgcacagtgcaaaaaaatgcaaaatacgtgtgtttagca

SEQ ID NO: 154 - DNA, 19 bp, Artificial Sequence
  Description of Artificial Sequence Synthetic oligonucleotide
cgctgtgtgtggggacaac

SEQ ID NO: 155 - DNA, 19 bp, Artificial Sequence
  Description of Artificial Sequence Synthetic oligonucleotide
tgccgtgtgcggggacaac

SEQ ID NO: 156 - DNA, 19 bp, Artificial Sequence
  Description of Artificial Sequence Synthetic oligonucleotide
gggctgcaaaggcttcttt

SEQ ID NO: 157 - DNA, 19 bp, Artificial Sequence
  Description of Artificial Sequence Synthetic oligonucleotide
gggctgcaagggctttttc

SEQ ID NO: 158 - DNA, 19 bp, Artificial Sequence
  Description of Artificial Sequence Synthetic oligonucleotide
gcgcacagtgcaaaaaaat

SEQ ID NO: 159 - DNA, 19 bp, Artificial Sequence
  Description of Artificial Sequence Synthetic oligonucleotide
gagaacagtgcagaaaaat

SEQ ID NO: 160 - DNA, 50 bp, Homo sapiens
ctgcctcttcattgcctgcttcgtcctggtcctcacgcagagctccatct

SEQ ID NO: 161 - DNA, 49 bp, Homo sapiens
ctgcctcatggttgcctgtccggtcctcatcctcacccagagctccatc

SEQ ID NO: 162 - DNA, 19 bp, Artificial Sequence
  Description of Artificial Sequence Synthetic oligonucleotide
cctcacccagagctccatc

SEQ ID NO: 163 - DNA, 19 bp, Artificial Sequence
  Description of Artificial Sequence Synthetic oligonucleotide
catggtctacttcaacttc

SEQ ID NO: 164 - DNA, 19 bp, Artificial Sequence
  Description of Artificial Sequence Synthetic oligonucleotide
gtctacttcaacttctttg

SEQ ID NO: 165 - RNA, 84 nt, Artificial Sequence
  Description of Artificial Sequence Synthetic oligonucleotide
gccuucuuuaucuuggaucuuggccuuaaaaaaaggagaugcguaggguggacugagugaugucugucaagaccucguccaaac

SEQ ID NO: 166 - RNA, 84 nt, Artificial Sequence
  Description of Artificial Sequence Synthetic oligonucleotide
gccuucuuuaucuuggaucuuggccuuaaaaaaaggaggguugcguugcaaguaacagaaaggaauuugaaaauuguggcaaac

SEQ ID NO: 167 - RNA, 84 nt, Artificial Sequence
  Description of Artificial Sequence Synthetic oligonucleotide
gccuucuuuaucuuggaucuuggccuuaaaaaaaggagacacaugugacaagaggcuaugauccugaaugcauccuuggcaaac

SEQ ID NO: 168 - RNA, 84 nt, Artificial Sequence
  Description of Artificial Sequence Synthetic oligonucleotide
gccuucuuuaucuuggaucuuggccuuaaaaaaaggaguagugcagcauugacugaaugucauacggcauaagcaucuacaaac

SEQ ID NO: 169 - RNA, 84 nt, Artificial Sequence
  Description of Artificial Sequence Synthetic oligonucleotide
gccuucuuuaucuuggaucuuggccuuaaaaaaaggaguauacauucuugguucauaaaggauaaggccuaagucgggucaaac

SEQ ID NO: 170 - RNA, 27 nt, Artificial Sequence
  Description of Artificial Sequence Synthetic oligonucleotide
gccuucuuuaucuuggaucuuggccuu

SEQ ID NO: 171 - RNA, 50 nt, Artificial Sequence
  Description of Artificial Sequence Synthetic oligonucleotide
ggagaugcguaggguggacugagugaugucugucaagaccucguccaaac

SEQ ID NO: 172 - RNA, 50 nt, Artificial Sequence
  Description of Artificial Sequence Synthetic oligonucleotide
ggaggguugcguugcaaguaacagaaaggaauuugaaaauuguggcaaac

SEQ ID NO: 173 - RNA, 50 nt, Artificial Sequence
  Description of Artificial Sequence Synthetic oligonucleotide
ggagacacaugugacaagaggcuaugauccugaaugcauccuuggcaaac

SEQ ID NO: 174 - RNA, 50 nt, Artificial Sequence
  Description of Artificial Sequence Synthetic oligonucleotide
ggaguagugcagcauugacugaaugucauacggcauaagcaucuacaaac

SEQ ID NO: 175 - RNA, 50 nt, Artificial Sequence
  Description of Artificial Sequence Synthetic oligonucleotide
ggaguauacauucuugguucauaaaggauaaggccuaagucgggucaaac

SEQ ID NO: 176 - DNA, 89 bp, Artificial Sequence
  Description of Artificial Sequence Synthetic oligonucleotide
gggagacaaacaaagagcgacaagggcagagtggacggtcggctagagccgggaggaattccttgtgaccacccagaagaagccagaag

SEQ ID NO: 177 - DNA, 50 bp, Homo sapiens
ccatgaactacatggtgtacttcaacttctttgcctgtgtgctggtgccc

SEQ ID NO: 178 - DNA, 47 bp, Homo sapiens
gcatggagtacatggtctacttcaacttctttgtgtgggtgctgccc

(19) United States
(12) Patent Application Publication
(10) Pub. No.: US 2024/0229032 A1
(43) Pub. Date: Jul. 11, 2024

(54) Multitargeting RNA Immunotherapy Compositions

(71) Applicant: SYSTEMS ONCOLOGY, LLC, Scottsdale, AZ (US)

(72) Inventors: Spyro Mousses, Scotsdale, AZ (US); David Azorsa, Scotsdale, AZ (US); Daniel Feldheim, Boulder, CO (US); James Heil, Boulder, CO (US); Necky Tran, London, Ontario (CA); Gregory Allen Penner, Toronto, Ontario (CA)

(21) Appl. No.: 18/558,945

(22) PCT Filed: May 5, 2022
(86) PCT No.: PCT/US2022/027902

§ 371 Date: Nov. 3, 2023

Related U.S. Application Data
(60) Provisional application No. 63/185,359, filed on May 6, 2021.
(60) Provisional application No. 63/231,234, filed on Aug. 9, 2021.
(60) Provisional application No. 63/242,865, filed on Sep. 10, 2021.
(60) Provisional application No. 63/250,548, filed on Sep. 30, 2021.
(60) Provisional application No. 63/287,037, filed on Dec. 7, 2021.
(60) Provisional application No. 63/287,040, filed on Dec. 7, 2021.
(60) Provisional application No. 63/323,997, filed on Mar. 25, 2022.

(51) Int. Cl. C12N 15/113; C12N 15/115

(52) U.S. Cl. CPC C12N 15/113; C12N 15/115; C12N 2310/14; C12N 2310/16; C12N 2310/3519; C12N 2310/51; C12N 2310/531; C12N 2320/32

(57) ABSTRACT

A multi-targeting siRNA-aptamer platform is provided that is processed by cellular RNAi machinery to produce siR-NAs. The constructs according to the invention are targeted at cells of the immune system. Methods of using the multi-targeting siR-NA-aptamer for selectively targeting cells to down-regulate the expression of multiple genes are also provided.

### RELATED AND PRIORITY APPLICATIONS

This application is a National Stage of International Application No. PCT/US2022/027902, filed May 5, 2022, which claims priority to U.S. Provisional Patent Application No. 63/185,359 filed May 6, 2021, U.S. Provisional Patent Application No. 63/231,234 filed Aug. 9, 2021, U.S. Provisional Patent Application No. 63/242,865 filed Sep. 10, 2021, U.S. Provisional Patent Application No. 63/250,548 filed Sep. 30, 2021, U.S. Provisional Patent Application No. 63/287,037 filed Dec. 7, 2021, U.S. Provisional Patent Application No. 63/287,040 filed Dec. 7, 2021 and U.S. Provisional Patent Application No. 63/323,997 filed Mar. 25, 2022. All of the above applications are incorporated herein by reference in their entirety.

### FIELD OF THE INVENTION

The invention is generally directed to RNA compositions for inhibiting gene expression in targeted immune cells.

### SEQUENCE LISTING

The instant application contains a Sequence Listing which has been submitted electronically in ASCII format and is hereby incorporated by reference in its entirety. Said ASCII copy, created on May 26, 2022, is named 10911_011095-WO0_SL.txt and is 42,027 bytes in size.

### BACKGROUND OF THE INVENTION

Cancer immunotherapy, or immuno-oncology, is a cancer treatment that engages the power of the body's immune system to treat cancer. Immunotherapy in its various forms may work by educating the immune system to recognize and attack cancer cells, enhance immune cells to promote the elimination of cancer cells, or by providing immune response enhancers.

Immuno-oncology agents may include targeted antibodies, cancer vaccines, adoptive cell transfer, tumor-infecting viruses, checkpoint inhibitors, cytokines, and adjuvants. The immune-oncology therapeutics according to certain aspects of the instant invention include multi-targeting RNA constructs.

RNA interference (RNAi), also known as RNA silencing, has been extensively explored for therapeutic use in reducing gene expression but in the decades since its discovery few therapeutics have been approved. The traditional design pattern for RNA inhibition is that one piece of siRNA aims at one specific sequence (Reynolds et al., Nat Biotechnol, 22:326-330 (2004)). There remains a need in the art for compositions and methods of delivering siRNA to cells (e.g., malignant cells, tumor-associated T cells, effector T cells) to inhibit diseases such as cancer, metastasis or metabolic diseases. The nucleic acid compounds and methods of using the same as provided herein solve these and other problems in the art.

Recent work has expanded the RNA constructs to include joining two siRNAs to inhibit two different targets (Liu et al., Sci Reports, 6: (2016)). SiRNA's processed by cellular RNAi machinery to produce two siRNAs as opposed to dual administration offers a number of benefits including increased circulating half-life and reduced renal excretion (Liu et al., Sci Reports, 6: (2016)).

Dual targeting of genes by a single siRNA through targeting conserved homologous regions has been shown to be effective to inhibit the expression of gene families by diminishing the function of escape pathways. In vitro, a multi-target siRNA targeting the conserved homology region of DNMT3 family members effectively inhibited expression (Du et al., Gen and Mol Bio, 35:164-171(2012)).

Delivery to tissues other than the liver has remained a complication and hinderance for RNAi therapies. Aptamer-siRNA chimeras have been used to effectively deliver siRNA's to downregulate expression of oncological genes targets (Liu et al., Sci Reports, 6: (2016)).

U.S. Pat. No. 6,506,559 discloses a method to inhibit expression of a target gene in a cell, the method comprising the introduction of a double-stranded RNA into the cell in an amount sufficient to inhibit expression of the target gene, wherein the RNA is a double-stranded molecule with a first ribonucleic acid strand consisting essentially of a ribonucleotide sequence which corresponds to a nucleotide sequence of the target gene and a second ribonucleic acid strand consisting essentially of a ribonucleotide sequence which is complementary to the nucleotide sequence of the target gene. Furthermore, the first and the second ribonucleotide strands are separately complementary strands that hybridize to each other to form the said double-stranded construct, and the double-stranded construct inhibits expression of the target gene.

U.S. Pat. No. 5,475,096 discloses nucleic acid molecules each having a unique sequence, each of which has the property of binding specifically to a desired target compound or molecule. Each nucleic acid molecule is a specific ligand of a given target compound or molecule. The process, known as SELEX, is based on the idea that nucleic acids have sufficient capacity to form a variety of two- and three-dimensional structures with sufficient chemical versatility available within their monomers to act as ligands (form specific binding pairs) with virtually any chemical compound, whether monomeric or polymeric. Molecules of potentially any size can serve as targets.

The SELEX method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

U.S. Pat. No. 9,953,131 discloses a method for designing a dual-targeting short interfering RNAs (siRNAs) in which both strands are deliberately designed to separately target different mRNA transcripts with complete complementarity. This approach is limited by the rarity of useful sequences of this type.

U.S. Pat. No. 9,777,278 discloses an interfering nucleic acid (iNA) duplex comprised of a sense strand of nucleotides having a 5′ end and a 3′ end annealed onto an antisense strand of nucleotides having a 5′ end and a 3′ end wherein the antisense strand has at least two segments, wherein one segment of the antisense strand can target a first RNA and another segment of the antisense strand can target a second RNA, or one segment of the antisense strand can target a first portion of an RNA and another segment of the antisense strand can target a second non-contiguous portion of said RNA.

U.S. Pat. No. 9,695,425 discloses an siRNA molecule that, when internalized by a B cell, suppresses expression of BAFF-R and one other target oncogene selected from: Bcl6, Bcl2, STAT3, Cyclin D1, Cyclin E2 and c-myc.

U.S. Pat. No. 10,689,654 discloses a bivalent siRNA-aptamer chimera capable of silencing two or more genes. Methods of using the bivalent siRNA chimeras for selectively targeting cells to down-regulate the expression of multiple genes is also disclosed (incorporated herein in its entirety by reference).

Du et al., Gen and Mol Bio, 35:164-171(2012) discloses a siRNA targeting the conserved homologous region of DNMT3 family members.

U.S. Pat. No. 10,689,654 discloses a bivalent siRNA-aptamer chimera platform that incorporates two aptamers for increase efficiency of delivering siRNAs to the targeted cell. Furthermore, those aptamers are conjugated to an siRNA construct that is processed by cellular RNAi machinery to produce at least two different siRNAs to inhibit expression of two or more different genes (incorporated herein in its entirety by reference).

U.S. patent application Ser. No. 15/899,473 discloses bispecific aptamers (incorporated herein in its entirety by reference).

U.S. Pat. No. 9,567,586 discloses an EPCAM aptamer coupled to an siRNA.

U.S. Pat. No. 10,385,343 discloses a method of treating cancer by administering a chimeric molecule comprising an EPCAM binding aptamer domain and an inhibitory nucleic acid domain that targets Plk1.

Patent Application PCT/US2020/038355 discloses an EpCAM-binding aptamer domain conjugated to an siRNA that inhibits the expression of a gene selected from the group consisting of: UPF2; PARP1; APE1; PD-L1; MCL1; PTPN2; SMG1; TREX1; CMAS; and CD47 for the purpose of treating cancer.

U.S. Pat. No. 10,960,086 discloses an siRNA-aptamer chimera that utilizes two aptamers targeting HER2 and HER3 and an siRNA targeting EGFR (incorporated herein in its entirety by reference).

U.S. Pat. No. 8,828,956 N-acetylgalactosamine (GalNAc)-siRNA conjugates that enables subcutaneous dosing of RNAi therapeutics with potent and durable effects and a wide therapeutic index. This delivery systems is only effective for delivering to the liver as GalNAc binds to the Asialoglycoprotein receptor (ASGPR) that is predominantly expressed on liver hepatocytes.

U.S. Pat. No. 8,058,069 discloses lipid nanoparticle (LNP) delivery technology. LNP technology (formerly referred to as stable nucleic acid-lipid particles or SNALP) encapsulates siRNAs with high efficiency in uniform lipid nanoparticles that are claimed to be effective in delivering RNAi therapeutics to disease sites in various preclinical models.

U.S. Pat. No. 10,278,986 discloses an antibody conjugated to an siRNA as a delivery mechanism. The antibody targets C5aR and the siRNA targets C5 expression for the treatment of rheumatoid arthritis. Patent Application PCT/US2020/036307 discloses a method of preparing an antibody covalently linked to one or more oligonucleotides.

Aptamers are single-stranded RNA or DNA oligonucleotides that are capable of binding with high affinity and specificity and are cost effective to produce. Aptamers are of great interest as an antibody-like replacement and are being investigate for their ability to selectively bind to a specific target, including proteins, peptides, carbohydrates, etc., as well as function as a ligand for directed drug delivery. However, there are two primary hurdles for aptamers reaching clinical significance, their need to be stabilized for in vivo use against nuclease degradation which results in a short half life, and their rapid renal clearance due to their small size.

Native DNA aptamers are more stable than RNA aptamers as RNA is a transient messenger. The in vitro half-life of an RNA aptamer in plasma is a few seconds, while a DNA aptamer has a half-life of up to hour (2000 White et al, 2002 Takei et al, 1991 Shaw et al). The 2′ hydroxyl group of RNA makes it chemically unstable, susceptible to hydrolysis, and allows for the catalysis of RNA strand scission by endoribonucleases (2009 Houseley et al). For these reasons, RNA aptamers are commonly chemically modified primarily at the 2′-position of pyrimidines to enhance stability.

U.S. Pat. No. 5,660,985 describes oligonucleotides containing nucleotide derivatives chemically modified at the 5- and 2′-positions of pyrimidines and purines including 2′-fluoro and 2′-amino modifications.

U.S. Pat. No. 5,580,737, describes highly specific nucleic acid ligands containing one or more nucleotides modified with 2′-amino (2′-NH_{#}), 2′-fluoro (2′-F), and/or 2′-O-methyl (2′-OMe).

U.S. patent application Ser. No. 08/264,029 describes oligonucleotides containing various 2′-modified pyrimidines.

U.S. patent application Ser. No. 10/524,817 describes a 4′-thioribonucleotide modified aptamer which was later developed in Kato, Y. et al. (2005) into an aptamer against human α-thrombin.

U.S. Pat. No. 9,914,914 describes six different modifications where the canonical ribofuranose ring of DNA and RNA is replaced by five- or six-membered congeners comprising HNA (1,5 anhydrohexitol nucleic acids), CeNA (cyclohexenyl nucleic acids), LNA (2′-O,4′-C-methylene-β-D-ribonucleic acids; locked nucleic acids), ANA (arabinonucleic acids), FANA (2′-fluoro-arabinonucleic acid) and TNA (a-L-threofuranosyl nucleic acids).

U.S. patent application Ser. No. 61/748,834 describes Threose nucleic acid (TNA) modified aptamers.

U.S. patent application Ser. No. 12/044,895 describes double-stranded locked nucleic acid modifications (2′-O,4′-C-methylene-β-D-ribonucleic acids).

Lato, S. M. (2002) Nucleic Acids Res., describes Ribonucleoside 5′-(alpha-P-borano)-triphosphates (BH₃-RNA) modified aptamers.

PCT Publication No. 1997/004726 describes spiegelmers which are mirror images of the natural aptamers in which the D-ribose (the natural ribose) are replaced with the unnatural L-ribose. PCT Publication NO. 2001/006014 describes one of the first SELEX generated spiegelmers developed against D-adenosine.

Jhaveri, S. et al. (1998) Bioorg. Med. Chem. Lett., describes Ribonucleoside 5′-(alpha-thio) triphosphates (S-RNA) modified aptamers.

PCT Publication NO. 1994/010562 describes RNA aptamers containing photoreactive chromophore 5-iodouridine using crosslinking SELEX.

The immune system plays an important role in protection from disease. T cells are part of the immune system that have a capacity to selectively recognize and kill pathogens (plus cancer cells) through a coordinated immune response.

Checkpoints in the immune system prevents the destruction of healthy cells during an immune response. Cancer cells sometimes exploit immune checkpoints to evade detection and elimination.

Various new drugs, including monoclonal antibodies targeting PD-1, PD-L1 and CTLA-4 allow the immune system to overcome a cancer's ability to resist the immune responses and stimulate the body's own mechanisms to remain effective in its defenses against cancer.

The PD-1 (programmed cell death-1) receptor is expressed on the surface of activated T cells. PD-1 and a variety of other checkpoints halt or limit the immune system's T cell response.

Immune checkpoint inhibition in cancer therapy has been shown to be effective for the treatment of a number of different types of cancer. However, not all cancers cells respond equally. Additionally, toxicity and the development of resistance to individual checkpoint inhibitors are problematic (Pardoll, 2012; Topalian et al., 2015). Improvements for immune checkpoint inhibitors are needed to combat aforementioned drawbacks.

The first immune-checkpoint inhibitor approved by the U.S. Food and Drug Administration (FDA) was ipilimumab, a fully human immunoglobulin G1 monoclonal antibody that blocks cytotoxic T-lymphocyte antigen (CTLA)-4 and consequently the PD-1 pathway for the treatment of metastatic melanoma in 2011. The finding that programmed cell death protein 1 ligand 1 (PDL1 or PD-L1) and PDL2 are expressed by melanoma cells, T cells, B cells and natural killer cells led to the development of programmed cell death protein 1 (PD1 or PD-1)-specific antibodies (e.g., nivolumab and pembrolizumab).

Thus, PD1 pathway blockade has become a major focus in anticancer drug development beyond melanoma. In addition to benefiting patients with renal cell carcinoma, it has benefit in patients with tumors previously not considered sensitive to immunotherapies, including non-small cell lung cancer. However, there are still limitations due to toxicity associated with these immunotherapies. Thus, there is a need for an immunotherapy blocking the PD1 pathway with the best balance of high efficacy and low toxicity.

Metastatic melanoma is an aggressive disease with a 16% 5-year survival rate and responds poorly to most standard chemotherapies. Interferon and interleukin 2 (IL-2) have both been approved by the U.S. Food and Drug Administration for the treatment of melanoma. Both mediate their benefit by stimulating an antitumor immune response.

Therefore, certain embodiments of the invention provide immunotherapies that have reduced off-target effects for the treatment of cancer including but not limited to, ovarian cancer, non-small-cell lung cancer, cervical cancer, colon cancer, prostate cancer, esophageal and stomach cancers, and breast cancer.

### PD-1

Programmed cell death protein 1 (PD-1), a member of the immunoglobulin superfamily, is an immune checkpoint protein expressed upon the surface of activated T and B cells. The receptor plays a role in the maintenance of immune tolerance, which is mediated through the binding of its ligands PD-L1 and PD-L2 that are expressed by a variety of cell types, including immune cells such as antigen presenting cells. Tumor cells evade immune surveillance, in part, through their expression of PD-L1. Immune checkpoint inhibitors that block PD-1/PD-L1 interactions augment T cell mediated anti-tumor immunity and have changed the landscape of cancer therapy. Since 2014, the Food and Drug Administration (FDA) has approved multiple anti-PD1 and anti-PD-L1 monoclonal antibodies for the treatment of solid tumors, with pembrolizumab being the first-in-class PD-1 inhibitor that became clinically available. While the majority of patients do not respond to PD-1 blockade, a subset of patients has achieved durable responses. Notably, agents that block immune checkpoints, including PD-1/PD-L1 have been studies in combination clinical studies with other chemotherapy, radiation, cancer vaccines and other checkpoint inhibitors. Based on the results of the Checkmate 227 and 9LA phase 3 studies that enrolled patients with non-small lung cancer (NSCLC), the Food and Drug Administration approved the combination of nivolumab and ipilimumab, monoclonal antibodies that recognize PD-1 and CTLA-4, respectively. Given that tumor-infiltrating lymphocytes (TIL), encounter immunosuppressive conditions in the tumor microenvironment, the efficacy of checkpoint inhibitors may be limited due to an inability to restore sufficient effector function to exhausted T cells that lack the ability to mediate tumor rejection.

Embodiments of the instant invention comprising aptamer-siRNA molecules can reverse T cell exhaustion and enhance T cell-mediated tumor rejection and offers and attractive alterative to the systemic administration of two individual antibody therapies, which can be associated with immune related adverse events. Hellmann et al. (2019) N Engl J Med. 381:2020-31.

### CTLA-4

T cell activation is followed by the upregulation of the immunoglobulin superfamily member, Cytotoxic T lymphocyte antigen-4 (CTLA-4). Engagement of the receptor with its ligands B7.1(CD80) and B7.2(CD86) found on the surface of antigen presenting cells, delivers an inhibitory signal, designed to help maintain immune tolerance and homeostasis. The phenotype of the CTLA-4 knockout mouse confirms that the receptor is a negative regulator of T cell activation, as the animals develop a fatal lymphoproliferative disorder 2-3 weeks after birth. In 2011, ipilimumab became the first checkpoint inhibitor approved by the Food and Drug Administration. The anti-CTLA-4 monoclonal antibody was approved for first- or second-line treatment of patients with advanced melanoma. It has since been approved for the treatment of several additional solid tumors. Ipilimumab, like the PD-1/PD-L1 inhibitors have been evaluated in combination clinical studies designed to leverage drug synergism and limit treatment associated toxicities. Like PD-1, high level of CTLA-4 express on the surface of conventional T cells in the tumor microenvironment is associated with a dysfunctional, or an exhausted state that impedes the ability of T cells to effectively mediate tumor rejection.

Embodiments of the present invention comprise CTLA-4 aptamer-siRNA combinations that minimize T cell exhaustion, increase T cell effector function, and reduce treatment related immune toxicities. Jacob J et al. Adv Pharmacol 2021

### LAG3

The extracellular region of the type 1 transmembrane protein, Lymphocyte (LAG 3/CD223) shares approximately 20% amino acid homology with the T cell expressed protein CD4. However, unlike CD4, the LAG3 intracellular lacks the cysteine motif that would enable the association with lymphocyte-specific protein tyrosine kinase (Lck). Like CD4, MHC Class Il is a known ligand of LAG3; however, as LAG3 also regulates the activity of CD8 T cells, it is likely that alternative ligands exist. Two potential LAG3 ligands include, Galectin-3, a known modulator of CD4 and CD8 T cell activity and sinusoidal endothelial cell lectin (LSECtin). LAG3 is an inhibitory receptor that functions to maintain immune tolerance and protect the host from autoimmunity. In the tumor microenvironment (TME), LAG3, along with other inhibitory receptors such as CTLA-4 and PD-1, is upregulated on T cells in response to immunosuppressive cells and soluble factors present in the TME. Tumor infiltrating lymphocytes progressive lose their ability to mediate tumor rejection and acquire and exhausted phenotype. Cancer immunotherapy approaches have been designed to restore T cell function and augment anti-tumor immunity. Presently, there are greater than 30 trials listed in the ClinicalTrials.gov database involving studies with anti-LAG-3 monoclonal antibodies. The majority of which are testing LAG3 antagonistic antibodies in combination with other immune checkpoint inhibitors. Creating aptamer-siRNA therapeutics would enable LAG3 inhibition in combination with other immune checkpoint inhibitors and provide a method for knocking down diverse inhibitory pathways that regulate functions such as inhibitory cytokine signaling and hypoxia, for example. Andrews L P et al. Immunol Rev 2017.

### CD73

The novel immunoinhibitory protein, CD73 (ecto-50-nucleotidase) contributes to tumor growth and metastasis. As an enzyme, CD73 works coordinately with CD39, which converts adenosine triphosphate (ATP) to adenosine diphosphate (ADP). CD73 then breaks down ADP and converts it to adenosine monophosphate (AMP). This process creates an immunosuppressive environment that limits the development of excessive immune responses in normal tissue. By interacting with T cell expressed Adenosine A2A receptors (A2AR) and Adenosine A2B receptors (A2BR) T cells bind immunosuppressive adenosine molecules. Tumors have exploited CD73-mediated adenosinergic mechanism to evade immune responses. In the tumor microenvironment (TME) CD73 and other adenosinergic molecules are inducible on cancer cells and immunosuppressive cell subsets that inhibit intratumoral T cell activity. In addition to the upregulation of CD73 on cancer cells, the protein is expressed on CD8+ T cells in the TME. Therefore, strategies to block the activity of CD73, CD39, or both, have been shown to boost CD8+ T cell responses in a tumor antigen-specific manner. Multiple CD73 antagonists, one small molecule inhibitor, AB680, and several anti-CD73 monoclonal antibodies are currently in early-stage clinical trials.

Embodiments of the instant invention comprising an RNA therapeutic to disrupt signaling through both CD79 and CD39 (on tumor cells or T cells) or inhibiting Adenosine A2AB-A2BR will limit immunosuppression in the TME. Nocentini A et al. Expert Opinion on Therapeutic Patents 2021. Roh M et al. Curr Opin Pharmacol 2020.

### VHL

Under nonmonic conditions, the hypoxia-inducible factors (HIFs), which are heterodimeric transcription factors, are constitutively degraded by a process that requires the von Hippel-Lindau (VHL) complex. The role of VHL as a tumor suppressor is well characterized and loss of VHL function due to spontaneous and inherited mutations causes VHL Syndrome that leads to renal and other specific cancers that may arise in multiple organs; however, HIFs play a role in immunity that may be regulated by VHL. Under conditions of low oxygen (hypoxia) VHL does not interact with the subunits HIF-1α and HIF-2α. As a result, HIF-1α and HIF-2α accumulate and, heterodimerization with HIF-1β and subsequent localization to the nucleus; that results in increased transcription of target genes that allow functional and metabolic adaptations to hypoxic microenvironments. HIF activity has been shown to influence T cell-mediated autoimmunity through the regulation of CD4+ regulatory T cells and TH17 helper T cells. However, the role of HIF-1α and HIF-2α in the differentiation and function of CD8+ T cells in vivo during the response to infection and cancer is poorly understood. Recently, it has been shown that VHL also intrinsically regulates CD8+ T cells. Knockout or knockdown of VHL in CD8+ T cells increases levels of HIF proteins in effector T cells and enables the effector cells to overcome T cell exhaustion, thereby enhancing antitumor immunity.

Embodiments of the instant invention include the administration of aptamer-siRNA molecules that target VHL in conventional T cells to enhance the effector function and boost T cell responses to cancer. Liikanen I et al. Journal of Clinical Investigation 2021.

### NR4A1

The nuclear receptor 4A1 (“nuclear receptor subfamily 4 group A member 1”) is a protein that in humans is encoded by the NR4A1 gene. NR4A1 is a member of the NR4A nuclear receptor family of intracellular transcription factors and is involved in cell cycle mediation, inflammation and apoptosis.

Nuclear receptor 4A1 plays a key role in mediating inflammatory responses in macrophages. In addition, subcellular localization of the NR4A1 protein plays a key role in the survival and death of cells.

Nr4a1 is strongly induced in thymocytes undergoing selection and controls the development of Treg cells. Nr4a1 also regulates the development and frequency of CD8+ T cells through direct transcriptional control of Runx3. Nr4a1 recruits the corepressor, CoREST to suppress Runx3 expression in CD8+ T cells. Loss of Nr4a1 results in increased Runx3 expression in thymocytes which causes an increase in the frequency and total number of intrathymic and peripheral CD8+ T cells. Nr4a1 is a critical player in the regulation of CD8 T cell development through the direct suppression of Runx3. Nowyhed, H., Huynh, T., Blatchley, A. et al. The Nuclear Receptor Nr4a1 Controls CD8 T Cell Development Through Transcriptional Suppression of Runx3. Sci Rep 5, 9059 (2015). https://doi.org/10.1038/srep09059. Thus the inhibition of Nr4A1 through RNA inhibition is a useful means of activating the immune system to fight cancer.

In spite of recent advances, there is a need in the art for compositions and methods of delivering modulators of cell activity (e.g., anti-tumor agents, anti-obesity agents) to cells (e.g., malignant cells, tumor-associated T cells, effector T cells) to inhibit diseases such as cancer, metastasis or metabolic diseases. The nucleic acid compounds and methods of using the same as provided herein solve these and other problems in the art.

### SUMMARY OF THE INVENTION

A multi-targeting siRNA-aptamer platform is provided that is processed by cellular RNAi machinery to produce siRNAs. The constructs according to the invention are targeted at cells of the immune system. Methods of using the multi-targeting siRNA-aptamer for selectively targeting cells to down-regulate the expression of multiple genes are also provided.

### BRIEF DESCRIPTION OF THE FIGURES

FIG. 1A: Depicts alignment of NR4A1, NR4A2 and NR4A3 gene sequences to identify multitargeting siRNA.

FIG. 1B: Depicts alignment of ADORA2A and ADORA2B gene sequences to identify dual targeting siRNA.

FIG. 2: Depicts effect of dual targeting siRNA treatment of cancer cells on ADORA2A and ADORA2B expression.

FIG. 3A: Depicts effect of gene specific siRNA treatment of cancer cells on NR4A1 expression normalized to GAPDH.

FIG. 3B: Depicts effect of gene specific siRNA treatment of cancer cells on NR4A2 expression normalized to GAPDH.

FIG. 3C: Depicts effect of gene specific siRNA treatment of cancer cells on NR4A3 expression normalized to GAPDH.

FIG. 4: Depicts effect of gene specific siRNA treatment of cancer cells on IDO1 and STAT3 expression normalized to GAPDH.

FIG. 5: Depicts effect of gene specific siRNA treatment of cancer cells on c-MYC and YY1 expression normalized to GAPDH.

FIG. 6A: Depicts effect of gene specific siRNA treatment of cancer cells on CBLB and TOX expression normalized to GAPDH.

FIG. 6B: Depicts effect of gene specific siRNA treatment of cancer cells on CBLB and TOX expression normalized to GAPDH.

FIG. 7: Depicts effect of gene specific siRNA treatment of cancer cells on RICTOR and TOX2 expression normalized to GAPDH.

FIG. 8: Depicts effect of gene specific siRNA treatment of cancer cells on UBC and VHL expression normalized to GAPDH.

FIG. 9: Depicts effect of gene specific siRNA treatment of cancer cells on ADORA2A and ADORA2B expression normalized to GAPDH.

FIG. 10: Depicts effect of gene specific siRNA treatment of cancer cells on PTPN2 and VHL expression normalized to GAPDH.

FIG. 11: Depicts effect of gene specific siRNA treatment of cancer cells on AKT1 and BATF expression normalized to GAPDH.

FIG. 12: Schematic depicting dual binding properties of bivalent aptamer-siRNA chimera.

FIGS. 13A-13E: Depict predicted folding structures of potential PD1 binding RNA aptamers.

FIGS. 14A-14C: Depict predicted folding structures of potential CTLA4 binding RNA aptamers.

FIGS. 15A-15C: Depict predicted folding structures of potential LAG3 binding RNA aptamers.

FIGS. 16A-16C: Depict predicted folding structures of potential TIM3 binding RNA aptamers.

FIG. 17A: Provides a table of 5-benzyl Uridine modified RNA aptamer sequences linked to UBB siRNA sequence via nucleotide linker.

FIG. 17B: Provides a table of 5-benzyl Uridine modified RNA aptamer sequences linked to UBB siRNA sequence via chemical linker.

FIG. 17C: Provides a reverse chimera structure using an alternative linker.

FIG. 18: Depicts data from animal studies supporting that NR4A1 is linked to CD8+ T cell dysfunction.

FIG. 19: Depicts transcription factors TOX and NR4A1 as master regulators of exhausted CD8+ T cell

FIG. 20: Depicts a bivalent aptamer targeting PD1 and CTLA4 that reverses exhaustion of T cells and helps T cells survive TME.

### DETAILED DESCRIPTION OF THE INVENTION

Cancer drugs are most effective when given in combination. One rationale for combination therapy is to use drugs that work by different mechanisms, thereby decreasing the likelihood that resistant cancer cells will develop. When drugs with different effects are combined, each drug can be used at its optimal dose, without intolerable side effects. See for example, https://www.merckmanuals.com/en-ca/home/cancer/prevention-and-treatment-of-cancer/combination-cancer-therapy, accessed May 3, 2021.

Combination therapy may also operate by simultaneously blocking two or more signaling pathways, Wu et al., Nat Biotechnol, 25:1290-1297 (2007). In addition, tumor progression and metastasis may be suppressed by overcoming the functional redundancy or synergistic action of targeted molecules (van der Veeken, et al., Current Cancer Drug Targets, 9:748-760 (2009)).

Zhao, et al. (Cancer discovery. 4. 10.1158/2159-8290.CD-13-0465, 2013) discuss the problem of intra-tumor heterogeneity and the approach of using computationally predictive combination therapy to address this problem.

According to certain embodiments of the instant invention constructs are provided which target cells of the immune system and deliver siRNAs that inhibit the expression of genes that prevent the immune system from effectively attacking and eliminating cancerous cells and tumors.

Preferred immune systems cellular targets according to certain embodiments of the invention are cell surface receptors on T cells.

Especially preferred immune systems cellular targets according to certain embodiments of the invention are PD-1, CTLA4, CD73, LAG3 and TIM-3.

Preferred siRNA target genes according to certain embodiments of the invention are those that suppress the immune system when activated.

Preferred siRNA target genes according to certain embodiments of the invention include checkpoint inhibitors.

Preferred siRNA target genes according to certain embodiments of the invention include NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E, or STAT3.

As used herein, the term “oncogene” refers to a gene that can in some circumstances transform a cell into a cancerous cell or a gene that promotes the survival of a cancer cell.

As used herein, the term “effective amount” in the context of the administration of a therapy to a subject refers to the amount of a therapy that achieves a desired prophylactic or therapeutic effect.

A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene (e.g. when expressed in the same cell as the gene or target gene). The complementary portions of the nucleic acid that hybridize to form the double stranded molecule typically have substantial or complete identity. In certain embodiments, a siRNA or RNAi is a nucleic acid that has substantial or complete identity to a target gene and forms a double stranded siRNA. In certain embodiments the “siRNA,” “small interfering RNA,” “small RNA,” or “RNAi” engages the cell's natural RNA-induced silencing complex “RISC” complex to silence genes.

In certain embodiments, the instant invention comprises a chimeric molecule including a cancer marker-binding domain and an inhibitory nucleic acid domain. As used herein, “cancer marker-binding domain” refers to a domain and/or molecule that can bind specifically to a molecule more highly expressed on the surface of a cancer-associated cell as compared to a healthy cell of the same type (a “cancer marker”). As used herein, “inhibitory nucleic acid domain” refers to a domain comprising an inhibitory nucleic acid. In some embodiments, the inhibitory nucleic acid can be a siRNA.

Certain embodiments of the instant invention comprise multi- and multi-multi-targeting siRNA and siRNA-targeting molecule chimeras in treating cancer and other diseases which can be treated by genetic inhibition. The compounds and methods in certain embodiments of the instant invention may utilize one or more aptamers that target the therapeutic constructs specifically to cancer cells, providing effective and on-target suppression of the gene or genes targeted by the siRNA.

As used herein “multi-multi-targeting siRNA or construct” refers to a set of unique and novel synthetic molecules for efficacious anti-tumor activity. These constructs each include siRNA molecules that each engage cell's RNA inhibition system to inhibit more than one different gene and that also include sequences found multiple times within each gene. Such multi-multi-targeting siRNA can be utilized alone or in constructs comprising multiple such siRNAs as well as one or more aptamers. Simple examples of such constructs can be targeted to one or more cancer cells and can inhibit or silence three or four genes although more exotic constructs can readily be envisioned by one skilled in the art once the instant invention is understood.

In certain embodiments, an aptamer-siRNA chimera according to the instant invention includes an aptamer targeting PD1 (NCBI Gene ID: 5133) PD1 is an immune checkpoint molecule exploited by tumors to dampen T cell activation and avoid autoimmunity and the effects of an inflammatory response. Inhibiting PD1 enhances anti-tumor immunity.

In certain embodiments, an aptamer-siRNA chimera according to the instant invention includes an aptamer targeting CTLA4 (NCBI Gene ID: 1493). CTLA4 is an immune checkpoint molecule whose expression is dysregulated in tumors and in tumor-associated T cells. (Santulli-Marotto, S. et al., Cancer Res 63:7483-7489 (2003)). U.S. patent application Ser. No. 16/892,995 provides a CTLA-4 aptamer conjugated to an antisense nucleic acid.

In certain embodiments, an aptamer-siRNA chimera according to the instant invention includes an aptamer targeting CD73 (NCBI Gene ID: 4907). CD73 is part of an enzyme cascade to breakdown ATP into adenosine. Overexpression of CD73 occurs in many cancers and leads to overproduction of adenosine which suppresses the antitumor immune response and helps aid cancer proliferation, angiogenesis, and metastasis.

In certain embodiments, an aptamer-siRNA chimera according to the instant invention includes an aptamer targeting LAG-3 (NCBI Gene ID: 3902). LAG3, cell surface molecule, is primarily expressed on activated Tcells and NK cells and is a marker for the activation of CD4+ and CD8+ T cells. The co-expression of LAG3 with other inhibitory molecules including PD-1 induces T cell exhaustion.

In certain embodiments, an aptamer-siRNA chimera according to the instant invention includes an aptamer targeting TIM-3 (NCBI Gene ID: 84868). TIM-3, cell surface molecule, is constitutively expressed on innate immune cells and suppresses innate antitumor immunity by mediating T-cell exhaustion. TIM-3 is co-expressed with PD-1 and is upregulated during PD-1 blocking therapy. Blocking the TIM-3 pathway enhances cancer immunity and increases interferon-gamma (IFN-γ) in T cells.

Chimeric molecules according to certain embodiments of the instant invention include aptamers directing a siRNA payload to a T cell associated with a tumor environment. Targets such as Pd-1, CTLA4, CD73, TIM-3 and LAG3 direct the payload to T cells in the tumor microenvironment that are associated with a dysfunctional, or an exhausted state that impedes the ability of T cells to effectively mediate tumor rejection.

In certain embodiments, the instant invention includes anti-CTLA-4 directed constructs of the that are useful for the treatment of patients with advanced melanoma as well as additional solid tumors. In addition, constructs may include additional aptamers that target and interfere with the PD-1/PD-L1 interaction to leverage drug synergism and limit treatment associated toxicities. Like PD-1, high level of CTLA-4 express on the surface of conventional T cells in the tumor microenvironment is associated with a dysfunctional, or an exhausted state that impedes the ability of T cells to effectively mediate tumor rejection so constructs according to embodiments of the instant invention can target both CTLA4 and PD-1 or PD-L1. Similarly, the multi-targeting constructs according to certain embodiments of the invention may target CD73, TIM-3 or LAG3.

T cell directed chimeric molecules according to certain embodiments of the instant invention as described may include a siRNA payload that, rather than killing the cell, further enhance the activation of the targeted immune cells. Thus siRNAs targeting NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E, and STAT3 are useful in certain embodiments of the instant invention.

In addition to aptamer targeting, certain embodiments include ligand-receptor analyses to prioritize specific siRNA targets in specific cell types within the tumor environment.

One embodiment provides a trivalent siRNA construct where one siRNA inhibits the expression of NR4A1, NR4A2 and NR4A3.

One embodiment provides a siRNA construct where one siRNA inhibits the expression of ADORA2A and ADORA2B.

One embodiment provides a siRNA construct where one siRNA inhibits the expression of TOX and TOX2.

In certain embodiments siRNAs have been experimentally verified by real-time RT-PCR analysis and shown to provide at least 70% target knockdown at the mRNA level when used under optimal delivery conditions (confirmed using validated positive control and measured at the mRNA level 24 to 48 hours after transfection using 100 nM siRNA).

In certain other embodiments, siRNAs have been demonstrated to silence target gene expression by at least 75% at the mRNA level when used under optimal delivery conditions as validated by positive controls and measured at the mRNA level 24 to 48 hours after transfection using 100 nM siRNA.

Another embodiment provides a siRNA-aptamer chimera.

In certain embodiments, an aptamer of the siRNA chimeras binds to a cell surface protein expressed on immune cells.

In certain embodiments, a method and constructs are provided that include administering to a subject in need thereof and effective amount of an RNA construct having at least one aptamer that specifically binds to Pd-1, CTLA4, CD73 or LAG3 and selected RNA that are processed to produce siRNA that inhibits expression of NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E, or STAT3.

Another embodiment provides a pharmaceutical composition containing one or more different bivalent siRNA chimeras in an amount effective to down down-regulate at least three different genes in a target cell.

The method includes administering a dual targeting siRNA agent to the subject to be treated. When the organism to be treated is a mammal such as a human, the composition can be administered by any means known in the art including, but not limited to oral or parenteral routes, including intravenous, intramuscular, subcutaneous, transdermal, and airway (aerosol) administration. In some embodiments, the compositions are administered by intravenous infusion or injection.

In certain embodiments, the instant invention is also designed for targeted delivery of the therapeutic constructs and thus rapid tumor treatment.

The inhibitory nucleic acid domain of constructs according to certain embodiments of the instant invention can inhibit the expression of a gene product that is upregulated in a cancer cell and/or the expression of a gene that is required for cell growth and/or survival. In some embodiments, the inhibitory nucleic acid domain can inhibit the expression of a gene selected from UBB (e.g. “Ubiquitin B”; NCBI Gene ID: 7314); UBC (e.g. “Ubiquitin C”; NCBI Gene ID: 7316), BCL2, STAT3, MYC, SYK, CCNE2, CCND1, CCND2, BIRC5, EGFR, UBB, UBC, NR4A1, NR4A2, NR4A3, ADORA2a, ADORA2b, ADORA1, MAP2K1, MAP2K2, MAPK3 (ERK1), MAPK1 (ERK2), HIF1, HIF2, PFKFB3, PFKFB4, PLK1, PLK4, CDK11A, CDK11B, CDK4, CDK6, PARP1, or PARP2. Sequences of these genes, e.g., the human mRNAs, may be obtained from the NCBI database and can be used according to the instant invention to inhibitory nucleic acids.

In certain embodiments, a dual-targeting siRNA targets NR4A1 (NCBI Gene ID: 3164) and NR4A2 (NCBI Gene ID: 4929). When T cells encounter sustained T cell stimulation through exposure to self-antigens, to chronic infections or to the tumor microenvironment, then effector T cells may become dysfunctional to avoid excessive immune responses, which is known as T-cell exhaustion. NR4A1, a driver of cancer cell survival, has been identified as a key mediator of T cell dysfunction and contributor of regulatory T-cell-mediated suppression of anti-tumor immunity in the tumor microenvironment. Nr4a2 is highly expressed in tumor-infiltrating cells than in bystander cells. Furthermore, mice lacking Nr4a1 and Nr4a2 genes specifically in Tregs showed resistance to tumor growth in transplantation models.

In certain embodiments, a dual-targeting siRNA targets NR4A1 and NR4A3 (NCBI Gene ID: 8013), which is expressed similarly to NR4A1.

In certain embodiments, a multi-targeting siRNA targets NR4A1, NR4A2, and NR4A3.

In certain embodiments, a dual-targeting siRNA targets ADORA2a (NCBI Gene ID: 135) and ADORA2b (NCBI Gene ID: 136). ADORA2a signaling during T cell activation strongly inhibited development of cytotoxicity and cytokine-producing activity in T cells, whereas the inhibition of T cell proliferation was only marginal. While an adenosine-rich environment may allow for the expansion of T cell, it impairs the functional activation of T cells. Targeting the ADORA2a immunosuppressive pathway restores both effector function and metabolic fitness of peripheral and tumor-derived CD8+ T cells. ADORA2b promotes the expansion of myeloid-deriver suppressor cells which are immunosuppressive cells that promote tumor progression by impairing antitumor T-cell responses and/or modulating angiogenesis. Experiments targeting both ADORA2a and aADORA2b have shown greater infiltration by CD8+ T cells as well as NK cells, and they encompass fewer Tregs.

In certain embodiments, a dual-targeting siRNA targets ADORA2a and ADORA1 (NCBI Gene ID: 134). ADORA1 and ADORA2A are paralogues and high-affinity receptors responding to low concentrations of extracellular adenosine.

In certain embodiments, a dual-targeting siRNA target TOX (NCBI Gene ID: 9760) and TOX2 (NCBI Gene ID: 84968). High-mobility group (HMG)-box transcription factors, TOX and TOX2, are critical for the transcriptional program of CD8+ T cell exhaustion downstream of NFAT.

In certain embodiments, a targeting construct targets YY1 (NCBI Gene ID: 7528) as one of the targets. Ying Yang 1, YY1 is a transcription factor that regulates transcriptional activation and repression of many genes associated malignant transformation. YY1 is known to me pro-tumorigenic in colon cancer.

In certain embodiments, a targeting construct targets CBLB (NCBI Gene ID: 868) as one of the targets. Cbl-b is expressed in all leukocyte subsets and regulates several signaling pathways in T cells, NK cells, B cells, and different types of myeloid cells.

In certain embodiments, a targeting construct targets BATF (NCBI Gene ID: 10538) as one of the targets. BATF, Basic Leucine Zipper ATF-Like Transcription Factor, plays a role in the development of different types of cancer, including colon cancer, lymphoma and multiple myeloma.

Certain embodiment this invention include siRNA targeting two genes or more genes selected from a list consisting of: NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E, and STAT3.

Certain embodiments of the instant invention include a linker as outline in U.S. Pat. No. 10,960,086. Alternative linkers can be substituted. 2-4 unpaired bases have been demonstrated to be sufficient to retain aptamer function. However, U's can be substituted in place of the A's. Additionally, in certain embodiments a streptavidin disulfide linker can be used (Ted et al., Nucleic Acid Research, 2006). The aptamers and siRNAs can be tethered to complementary linker sequences and hybridized together through Watson-Crick base pairing (Pastor et al., Mol Ther, 2011). Additionally, siRNA and aptamers can be tethered through a 4 nt (CUCU) linker or covalently fused through 2 nt linker (UU) (Zhou et al, Mol Ther, 2008) (Zhou et el., Theranostics, 2018). The aptamers and siRNAs can also be bound through a “sticky bridge” of 16 nt repeating GC with a three carbon spacer on either side of the sticky bridge (Zhou et al., Nucleic Acids, 2009). The aptamers and siRNAs can be conjugated with an acid-labile linkage or a kissing loop interaction (Huang et al., Chembiochem. 2009) (Guo et al., Human Gene Therapy, 2005).

In some embodiments, the methods described herein relate to treating a subject having or diagnosed as having cancer with a composition as described herein. Subjects having cancer can be identified by a physician using current methods of diagnosing cancer.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

In further embodiments, administration of a dual targeting siRNA agent is administered in combination an additional therapeutic agent. The dual targeting siRNA agent and an additional therapeutic agent can be administered in combination in the same composition, e.g., parenterally, or the additional therapeutic agent can be administered as part of a separate composition or by another method described herein.

The following non-limiting examples illustrate embodiments of the invention in operation. Having read the specification claims and Figures in their entirety, one skilled in the art will reasonably appreciate that numerous modifications and substitutions of the embodiments are possible and can be carried out without requiring undue experimentation. Such modifications and substitutions constitute part of the present invention.

### EXAMPLES

### Example 1: Targets for T Cell Directed RNAs

siRNAs directed against TIGIT, LAG3, HAVCR2, PDCD1, CTLA4, and NT5E were identified. These siRNAs target the sequences as outlined below and are useful in embodiments of the instant invention that target T cells.

| | |
|---|---|
| TIGIT | GAAGAAAGCCCUCAGAAUC SEQ ID NO: 1 |
| (NM_173799) | GUGCCGAGCUGCAUGACUA SEQ ID NO: 2 |
| | GCACAGCAGUCAUCGUGGU SEQ ID NO: 3 |
| | UCGCUGACCGUGAACGAUA SEQ ID NO: 4 |
| LAG3 | CGACUUUACCCUUCGACUA SEQ ID NO: 5 |
| (NM_002286) | CUACAGAGAUGGCUUCAAC SEQ ID NO: 6 |
| | CAACUCCCUUGACAGUGUA SEQ ID NO: 7 |
| | UGAGGUGACUCCAGUAUCU SEQ ID NO: 8 |
| HAVCR2 | AAAGGGAUGUGAAUUAUUG SEQ ID NO: 9 |
| (NM_032782) | GAAAACAUCUAUACCAUUG SEQ ID NO: 10 |
| | GAGCGGAGGUCGGUCAGAA SEQ ID NO: 11 |
| | CAGCAACCCUCACAACCUU SEQ ID NO: 12 |
| PDCD1 | GGGCGUGACUUCCACAUGA SEQ ID NO: 13 |
| (NM_005018) | CGGAGAGCUUCGUGCUAAA SEQ ID NO: 14 |
| | ACAAUAGGAGCCAGGCGCA SEQ ID NO: 15 |
| | GCAAUGACAGCGGCACCUA SEQ ID NO: 16 |
| CTLA4 | GAACCCAGAUUUAUGUAAU SEQ ID NO: 17 |
| (NM_001037631) | GAACCUCACUAUCCAAGGA SEQ ID NO: 18 |
| | AUAUAAAGUUGGAUGCGGA SEQ ID NO: 19 |
| | CUAGGAAGCUCCAGUUCGA SEQ ID NO: 20 |
| NT5E | GAACCUGGCUGCUGUAUUG SEQ ID NO: 21 |
| (NM_002526) | GGAAGUCACUGCCAUGGAA SEQ ID NO: 22 |
| | UGAAAUCACUGCAUUACAA SEQ ID NO: 23 |
| | GGACUUUAUUUGCCAUAUA SEQ ID NO: 24 |

### Example 2: Identification of Multi-Targeting Domains

Novel sequences were identified with highly conserved homology useful for dual or tiple targeting.

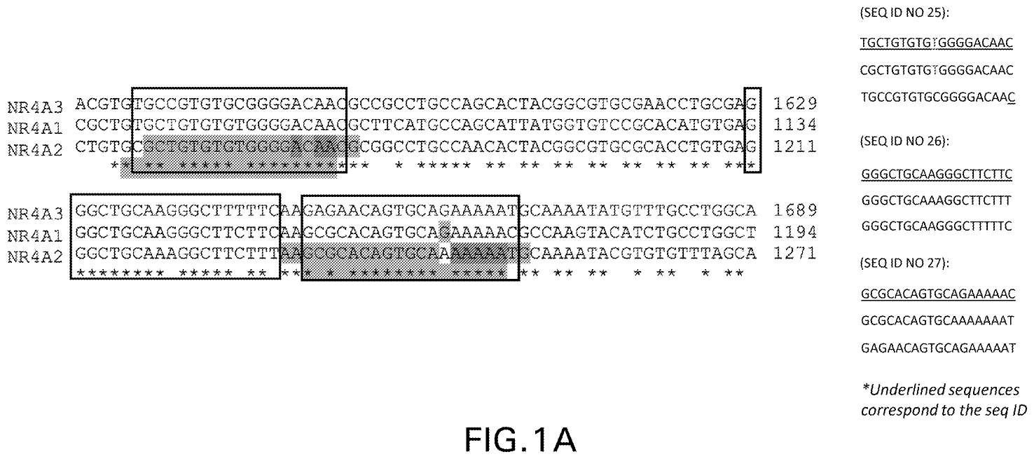

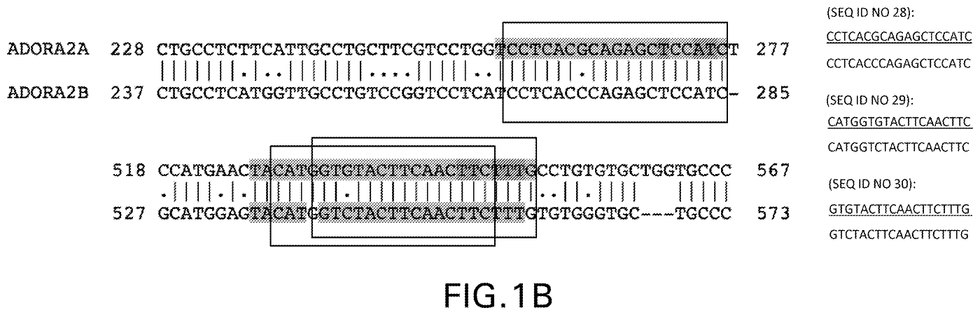

NR4A3 was found to have three targeting regions which have 18/19 conserved identities across all three sequences with NR4A1, and 18/19, 18/19, and 17/19 conserved identities with NR4A2 (FIG. 1A).

NR4A1, NR4A2, and NR4A3 siRNA targeting sequences:
(SEQ ID NO: 25):
5′-TGCTGTGTGTGGGGACAAC-3′
(SEQ ID NO: 26):
5′-GGGCTGCAAGGGCTTCTTC-3′
(SEQ ID NO: 27):
5′-GCGCACAGTGCAGAAAAAC-3′

ADORA2A was found to have three targeting regions which have 18/19 conserved identities across all three sequences with ADORA2B (FIG. 1B).

ADORA2A and ADORA2B siRNA targeting sequences:
(SEQ ID NO: 28):
5′-CCTCACGCAGAGCTCCATC-3′ (D04)
(SEQ ID NO: 29):
5′-CATGGTGTACTTCAACTTC-3′ (D05)
(SEQ ID NO: 30):
5′-GTGTACTTCAACTTCTTTG-3′ (D06)

### Example 3: siRNA Target Validation—Gene Expression Following siRNA Treatment

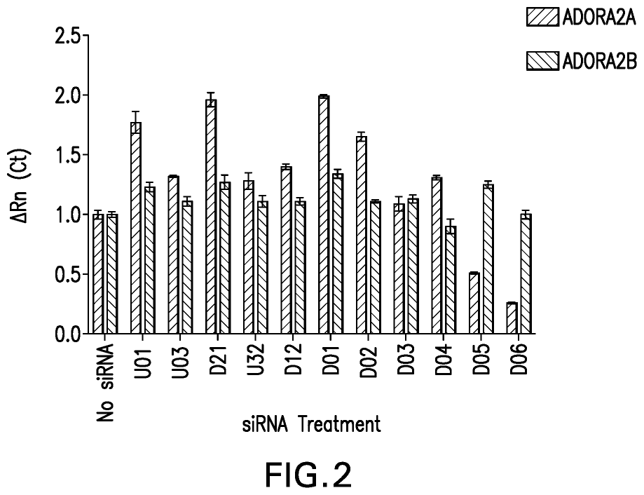

SKBR3 cells were treated with siRNA and the expression levels of ADORA2A/ADORA2B (FIG. 2) were measured. siRNA targeting (SEQ ID NO: 29) and (SEQ ID NO: 30) demonstrated the largest decrease in ADORA2A expression.

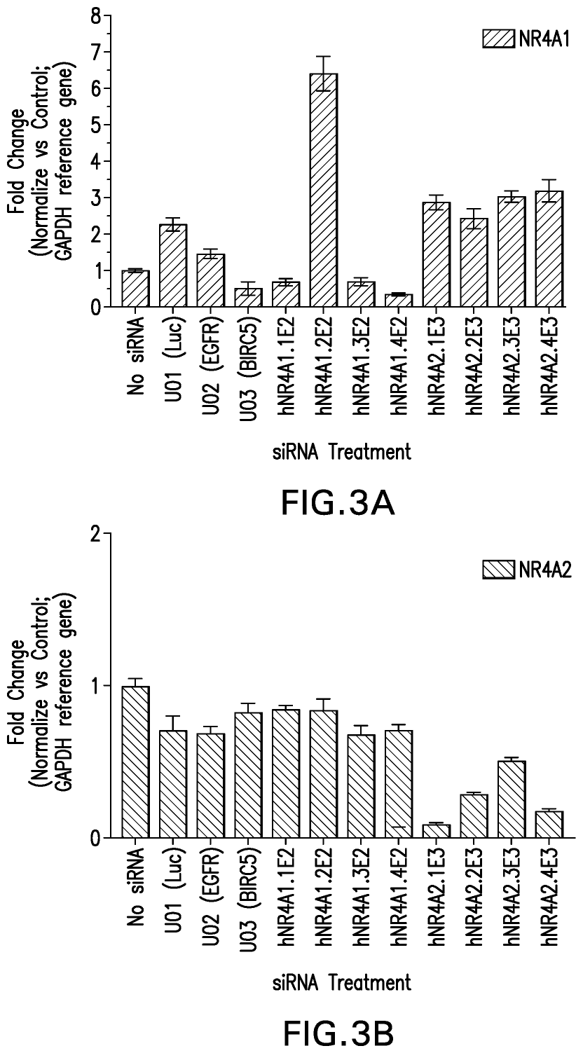

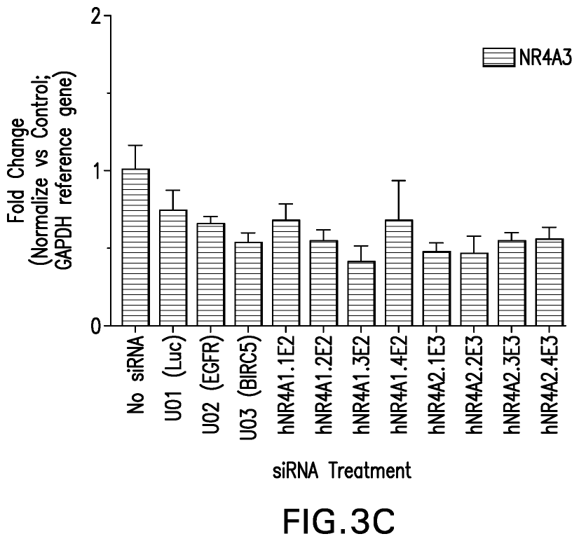

SK-BR3 cells were treated with siRNA and the expression of NR4A1(FIG. 3A), NR4A2 (FIG. 3B), and NR4A3 (FIG. 3C) was measured after treatment.

Target sequences of NR4A1:
(SEQ ID NO: 31):
5′-GCACCTTCATGGACGGCTA-3′ (hNR4A1.1E2)
(SEQ ID NO: 32):
5′-GCATTATGGTGTCCGCACA-3′ (hNR4A1.2E2)
(SEQ ID NO: 33):
5′-TGAAGGAAGTTGTCCGAAC-3′ (hNR4A1.3E2)
(SEQ ID NO: 34):
5′-CTGCAGAACCGCATCGCCA-3′ (hNR4A1.4E2)
Target sequences of NR4A2:
(SEQ ID NO: 35):
5′-CCACGTGACTTTCAACAAT-3′ (hNR4A2.1E3)
(SEQ ID NO: 36):
5′-ACATTCAGATGCACAACTA-3′ (hNR4A2.2E3)
(SEQ ID NO: 37):
5′-GGACAAGCGTCGCCGGAAT-3′ (hNR4A2.3E3)
(SEQ ID NO: 38):
5′-CCACCTTGCTTGTACCAAA-3′ (hNR4A2.4E3)

siRNA targeting (SEQ ID NO: 32) induced NR4A1 expression while (SEQ ID NO: 31), (SEQ ID NO: 33) and (SEQ ID NO: 34) reduced it. All four siRNAs targeting NR4A2 sequences reduced NR4A2 expression with (SEQ ID NO: 35) decreasing expression 91%. Sequences were found to moderately reduce NR4A3 expression.

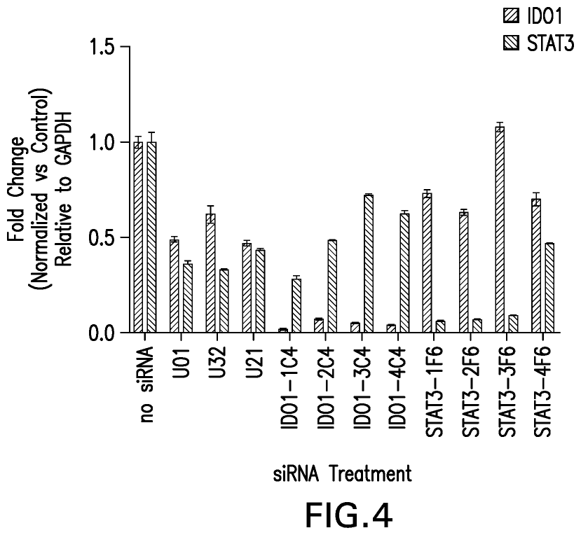

BT549 cells were treated with siRNA and the expression of STAT3 (FIG. 4) was measured after treatment.

Target sequences of STAT3:
(SEQ ID NO: 39):
5′-GGAGAAGCATCGTGAGTGA-3′ (STAT3-1F6)
(SEQ ID NO: 40):
5′-CCACTTTGGTGTTTCATAA-3′ (STAT3-2F6)
(SEQ ID NO: 41):
5′-TCAGGTTGCTGGTCAAATT-3′ (STAT3-3F6)
(SEQ ID NO: 42):
5′-CGTTATATAGGAACCGTAA-3′ (STAT3-4F6)

siRNAs targeting (SEQ ID NO: 39), (SEQ ID NO: 40), and (SEQ ID NO: 41) demonstrated decrease in STAT3 expression.

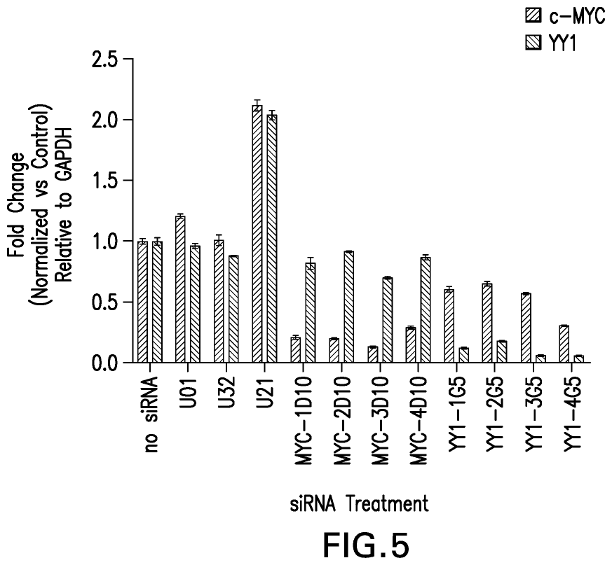

HCT116 cells were treated with siRNA and the expression of YY1 (FIG. 5) was measured after treatment.

Target sequences of YY1:
(SEQ ID NO: 43):
5′-GGATAACTCGGCCATGAGA-3′ (YY1-1G5)
(SEQ ID NO: 44):
5′-CAAGAAGAGTTACCTCAGC-3′ (YY1-2G5)
(SEQ ID NO: 45):
5′-GAACTCACCTCCTGATTAT-3′ (YY1-3G5)
(SEQ ID NO: 46):
5′-GCTTAGTAATGCTACGTGT-3′ (YY1-4G5)

All four siRNAs targeting YY1 also demonstrated decrease in expression levels, with (SEQ ID NO: 45) and (SEQ ID NO: 46) showing the largest reduction in expression.

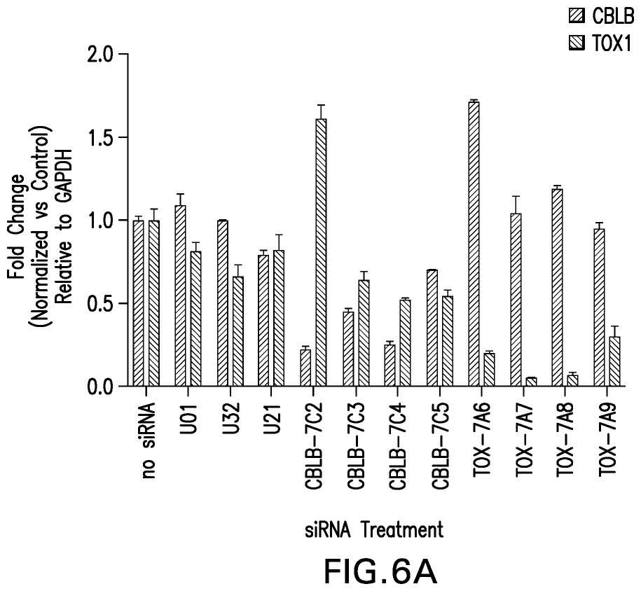

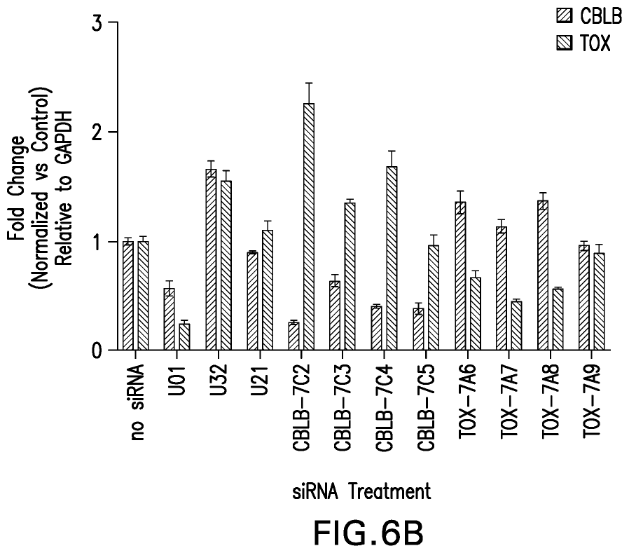

U2OS (FIG. 6A) and ES-2 (FIG. 6B) cells were treated with siRNA and the expression of CBLB and TOX was measured after treatment.

Target sequences of CBLB:
(SEQ ID NO: 47):
5′-GACCATACCTCATAACAAG-3′ (CBLB-7C2)
(SEQ ID NO: 48):
5′-TGAAAGACCTCCACCAATC-3′ (CBLB-7C3)
(SEQ ID NO: 49):
5′-GATGAAGGCTCCAGGTGTT-3′ (CBLB-7C4)
(SEQ ID NO: 50):
5′-TATCAGCATTTACGACTTA-3′ (CBLB-7C5)
Target sequences of TOX:
(SEQ ID NO: 51):
5′-CCACATGGCCAGCTGACTA-3′
(SEQ ID NO: 52):
5′-CAACCCGACTATCAGACTA-3′
(SEQ ID NO: 53):
5′-GAATGAATCCTCACCTAAC-3′
(SEQ ID NO: 54):
5′-GCAACAAGTTTGACGGTGA-3′

siRNAs targeting (SEQ ID NO: 47): demonstrated significant reduction in CBLB expression, but all four siRNAs showed efficacy. All four siRNAs targeting TOX demonstrated decreases in expression levels with (SEQ ID NO: 52) exhibiting the greatest expression decrease.

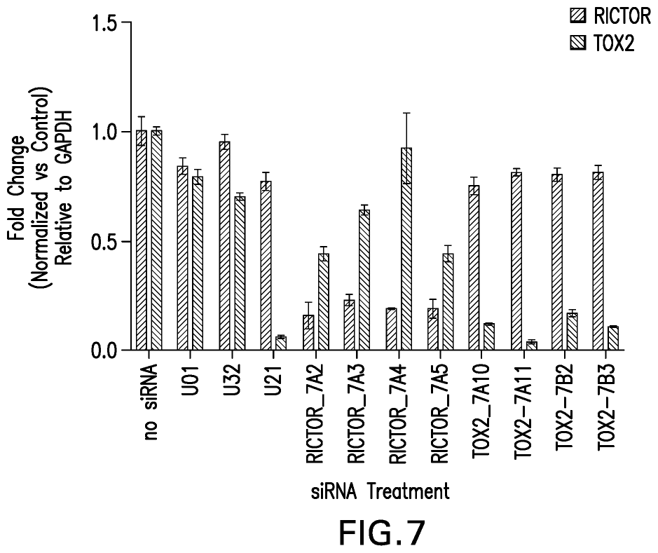

HCT116 cells were treated with siRNA and the expression of TOX2 (FIG. 7) was measured after treatment.

Target sequences of TOX2:
(SEQ ID NO: 55):
5′-GGAAGTGCATTTCAAGATC-3′ (TOX2_7A10)
(SEQ ID NO: 56):
5′-CGAGAACAACGAAGACTAT-3′ (TOX2_7A11)
(SEQ ID NO: 57):
5′-CAAGAGCACTCAGGCAAAC-3′ (TOX2_7B2)
(SEQ ID NO: 58):
5′-AAAGAGACCTTCAGCCGAC-3′ (TOX2_7B3)

All four siRNAs targeting TOX2 also demonstrated decreases in expression levels of TOX2 with (SEQ ID NO: 56) exhibiting the greatest expression decrease.

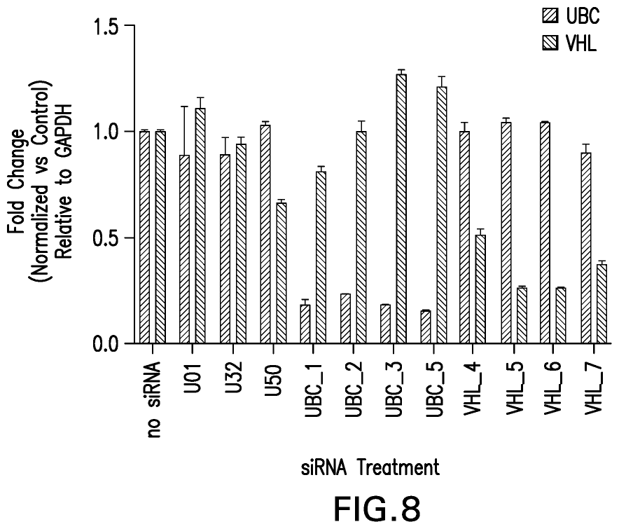

HCT116 cells were treated with siRNA and the expression of VHL (FIG. 8) was measured after treatment.

Target sequences of VHL:
(SEQ ID NO: 59):
5′-AAGGAGGTTTGTATAAGTAAT-3′ (VHL_4)
(SEQ ID NO: 60):
5′-CAGGAGCGCATTGCACATCAA-3′ (VHL_5)
(SEQ ID NO: 61):
5′-TTCAGTGGGAATTGCAGCATA-3′ (VHL_6)
(SEQ ID NO: 62):
5′-CTGATGAGTCTTGATCTAGAT-3′ (VHL_7)

All four siRNAs targeting VHL also demonstrated decreases in expression levels of VHL particularly (SEQ ID NO: 60) and (SEQ ID NO: 61).

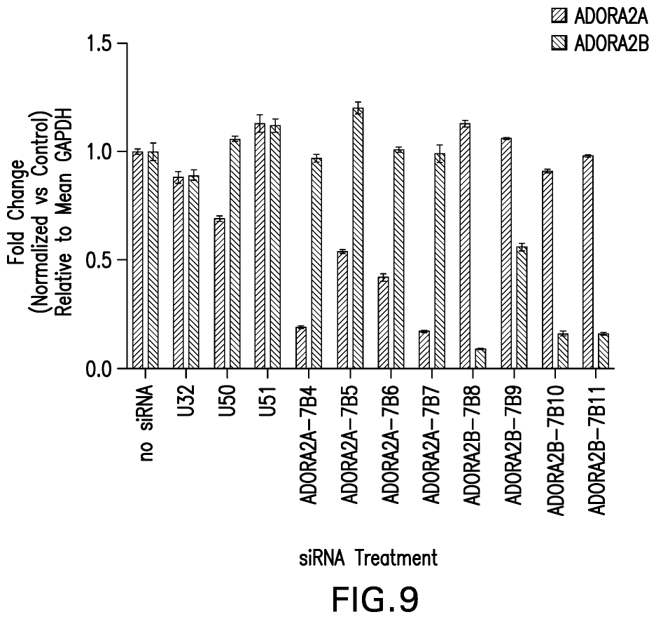

SKBR3 cells were treated with siRNA and the expression of ADORA2A and ADORA2B (FIG. 9) was measured after treatment.

Target sequences of ADORA2A:
(SEQ ID NO: 63):
5′-GAACGUCACCAACUACUUU-3′ (ADORA2A-7B4)
(SEQ ID NO: 64):
5′-CAUGCUGGGUGUCUAUUUG-3′ (ADORA2A-7B5)
(SEQ ID NO: 65):
5′-CAACUGCGGUCAGCCAAAG-3′ (ADORA2A-7B6)
(SEQ ID NO: 66):
5′-CCAAGUGGCCUGUCUCUUU-3′ (ADORA2A-7B7)
Target sequences of ADORA2B:
(SEQ ID NO: 67):
5′-UGAGCUACAUGGUAUAUUU-3′ (ADORA2b-7B8)
(SEQ ID NO: 68):
5′-GGGAUGGAACCACGAAUGA-3′ (ADORA2b-7B9)
(SEQ ID NO: 69):
5′-GAUGGAACCACGAAUGAAA-3′ (ADORA2b-7B10)
(SEQ ID NO: 70):
5′-GAACCGAGACUUCCGCUAC-3′ (ADORA2b-7B11)

All four siRNAs targeting ADORA2A demonstrated significant reduction in ADORA2A expression, with (SEQ ID NO: 63) and (SEQ ID NO: 65) demonstrating the most significant reduction in expression. All four siRNAs targeting ADORA2B also demonstrated decreases in expression levels of ADORA2B particularly (SEQ ID NO: 67).

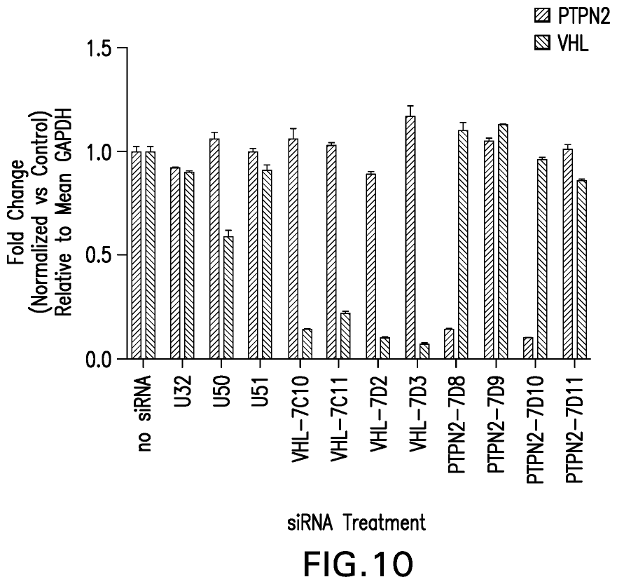

HCT116 cells were treated with siRNA and the expression of PTPN2 and VHL (FIG. 10) was measured after treatment.

Target sequences of PTPN2:
(SEQ ID NO: 71):
5′-GAAACAGGAUUCAGUGUGA-3′ (PTPN2-7D8)
(SEQ ID NO: 72):
5′-ACAAAGGAGUUACAUCUUA-3′ (PTPN2-7D9)
(SEQ ID NO: 73):
5′-AAAGGGAGAUUCUAGUAUA-3′ (PTPN2-7D10)
(SEQ ID NO: 74):
5′-AAACAGAAAUCGAAACAGA-3′ (PTPN2-7D11)
Target sequences of VHL:
(SEQ ID NO: 75):
5′-CCGUAUGGCUCAACUUCGA-3′ (VHL-7C10)
(SEQ ID NO: 76):
5′-AGGCAGGCGUCGAAGAGUA-3′ (VHL-7C11)
(SEQ ID NO: 77):
5′-GCUCUACGAAGAUCUGGAA-3′ (VHL-7D2)
(SEQ ID NO: 78):
5′-GGAGCGCAUUGCACAUCAA-3′ (VHL-7D3)

All four siRNAs targeting VHL demonstrated significant reduction in VHL expression with (SEQ ID NO: 78) demonstrating the most significant reduction in expression. Two siRNAs targeting PTPN2 also demonstrated significant in expression levels of PTPN2 particularly (SEQ ID NO: 73).

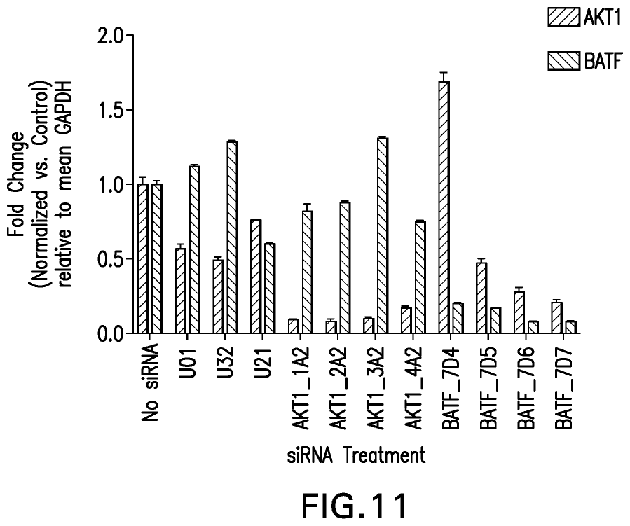

SKBR3 cells were treated with siRNA and the expression of BATF (FIG. 11) was measured after treatment.

Target sequences of BATF:
(SEQ ID NO: 79):
5′-GUACAGCGCCCACGCAUUC-3′ (BATF_7D4)
(SEQ ID NO: 80):
5′-GAAACAGAACGCGGCUCUA-3′ (BATF_7D5)
(SEQ ID NO: 81):
5′-GAACGCGGCUCUACGCAAG-3′ (BATF_7D6)
(SEQ ID NO: 82):
5′-AGAGUUCAGAGGAGGGAGA-3′ (BATF_7D7)

All four BATF targeting siRNAs exhibited significant reduction in BATF expression with possible dual action inhibition to AKT1.

### Example 4: Bivalent Aptamer-Driven Delivery of Two siRNAs

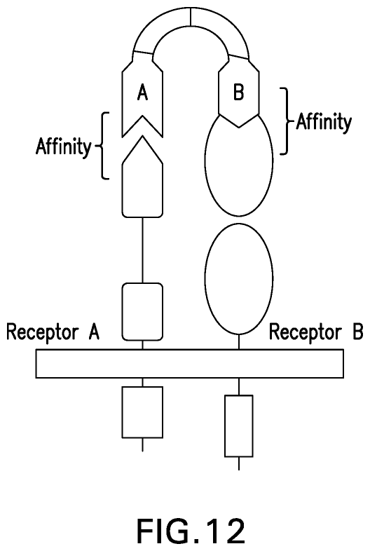

Bivalent aptamers support increased cargo internalization and specificity. Moreover, proof of concept experiments for increasing ligand valency to augment cargo delivery has been demonstrated by the use of nanoparticle-based carriers (Pardella et al., Cancers 2020, 12(10), 2799) (FIG. 12).

### Example 5: PD-1-NR4A1-VHL-CTLA4 Construct

Three RNAs are generated by in vitro transcription, with PCR products as templates.
RNA1: PD-1 aptamer-NR4A1 antisense siRNA
RNA2: CTLA4 aptamer and VHL sense siRNA and NR4A1 sense siRNA
RNA3: VHL anti-sense strand

Anti-PD1 Aptamer Sequences:
(SEQ ID NO: 83):
5′-GGCUGGGGUGCGACAUUAUGUUCGUUAAGGAUCAAUCGCUC-3′
(SEQ ID NO: 84):
5′-UUAUGAUGCAAAAACGAACUGGAAUGGCCAUGCAGGUACA-3′
(SEQ ID NO: 85):
5′-GGUUGGGGUGCGACAUUAUGUUCGUUAAGGAUCAAUCGCUC-3′
(SEQ ID NO: 86):
5′-GAUUUGGAGAGCAUUAUGUUAGGUUAAGGAUCAAUCUUCUA-3′
(SEQ ID NO: 87):
5′-GGCUGGGGUGCGACAUUAUGUUCGUUAAGGAUCAAUCGCUU-3′
SEQ ID NO: 88):
5′-GGGGUAAACGAACAGGAUUUUUUUAACGAGCUAUAUUGUUUCCUGU
UGCCCGUCCGUCUAGUCGUGAAGAGAGCAAGGUUACU-3′ (10B-1)
(SEQ ID NO: 89):
5′-GGGGUAAACGAACAGGACGACGGGUCGAAGCUGAAUAGGUAACCAA
UCACGGCAUAACUAGUCGUGAAGAGAGCAAGGUUACU-3′ (10B-10)
(SEQ ID NO: 90):
5′-GGGGUAAACGAACAGGAUGAGGGAGCAAAAAGGGCGAAAAUGCAGU
AACUAAACGUUCUAGUCGUGAAGAGAGCAAGGUUACU-3′ (10B-14)
(SEQ ID NO: 91):
5′-GGGGUAAACGAACAGGAUUUUUUUAACGAGCUAUAUUAUUUCCUGU
UGCCCGUCCGUCUAGUCGUGAAGAGAGCAAGGUUACU-3′ (10B-68)
(SEQ ID NO: 92):
5′-GGGGUAAACGAACAGGAACCAUUAAAUCAUAAGGAGAAAGAUGAUG
UGCGCGACAUAACUAGUCGUGAAGAGAGCAAGGUUACU-3′ (10B-84)
2_pyridyl Modification:
(SEQ ID NO: 93):
5′-GGGAGACAAACAAAGAGCGACAAGGGCAGCGGGUUAUCACGUUGGG
AACGGGCCAUCAACUCUUCUCACCACCCAGAAGAAGCCAGAAG-3′
(SEQ ID NO: 94):
5′-GGGAGACAAACAAAGAGCGACAAGGGCAGUAGUGAGGGAUUCACCA
GAGUGAAUGCGCUCCUCGGAAAUCACCCAGAAGAAGCCAGAAG-3′
(SEQ ID NO: 95):
5′-GGGAGACAAACAAAGAGCGACAAGGGCAGAACGGGCAAUGUCCAAG
GUGAGGCAGUUUGUAUGGACACACACCCAGAAGAAGCCAGAAG-3′
Benzyl Modification:
(SEQ ID NO: 96):
5′-GGGAGACAAACAAAGAGCGACAAGGGCAGUUGAGAUUGAGGAGUCA
GACCUGCGUCUCUAGUAACAAUGCACCCAGAAGAAGCCAGAAG-3′
(SEQ ID NO: 97):
5′-GGGAGACAAACAAAGAGCGACAAGGGCAGAGUGGACGGUCGGCUAG
AGCCGGGAGGAAUUCCUUGUGACCACCCAGAAGAAGCCAGAAG-3′
(SEQ ID NO: 98):
5′-GGGAGACAAACAAAGAGCGACAAGGGCAGUUUGACAAUGUACCUUU
AAUUACGGAUUGUACCUUGGGCGCACCCAGAAGAAGCCAGAAG-3′

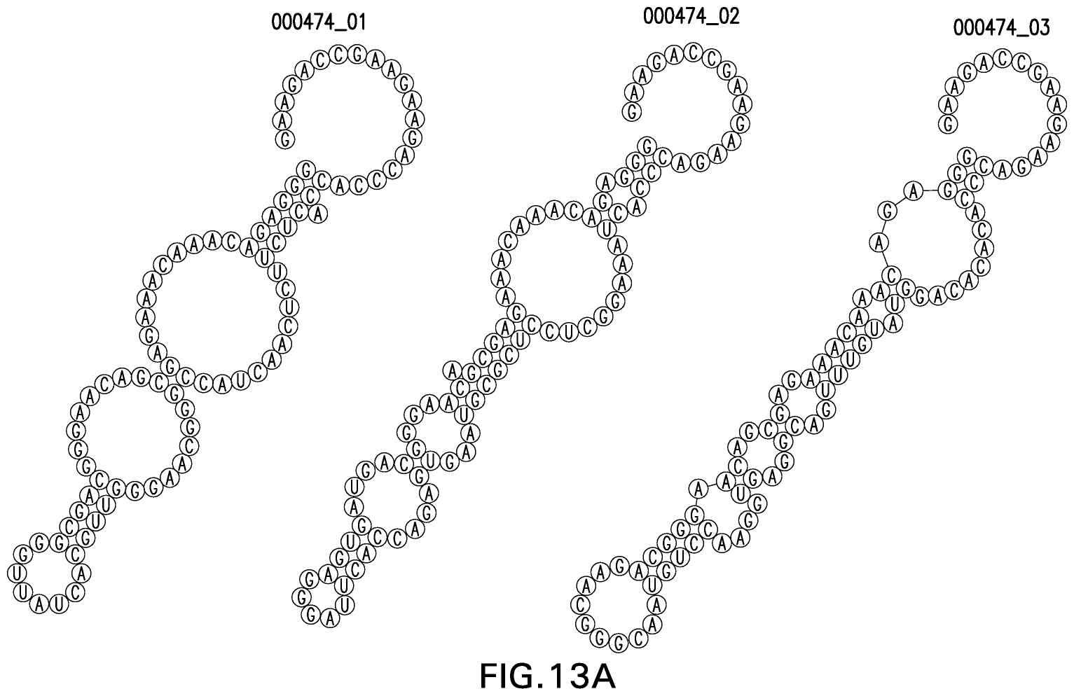

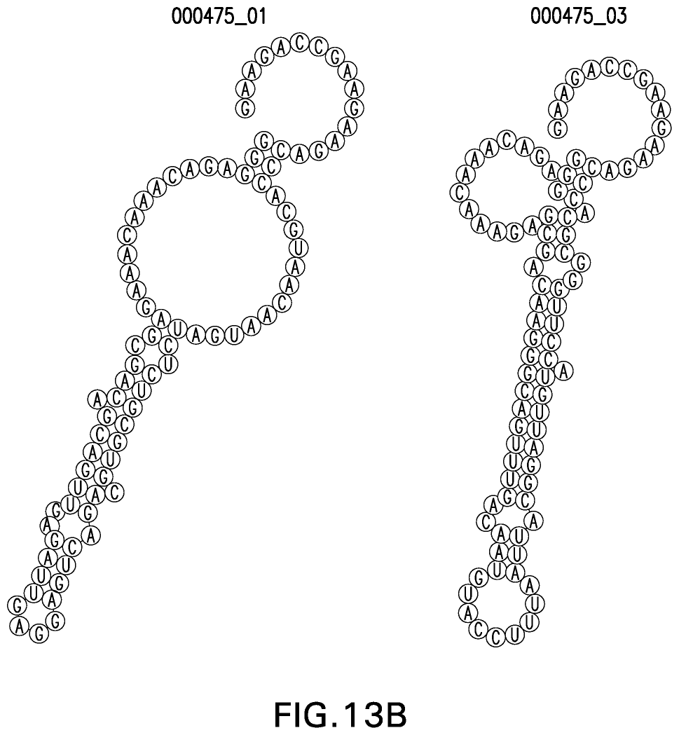

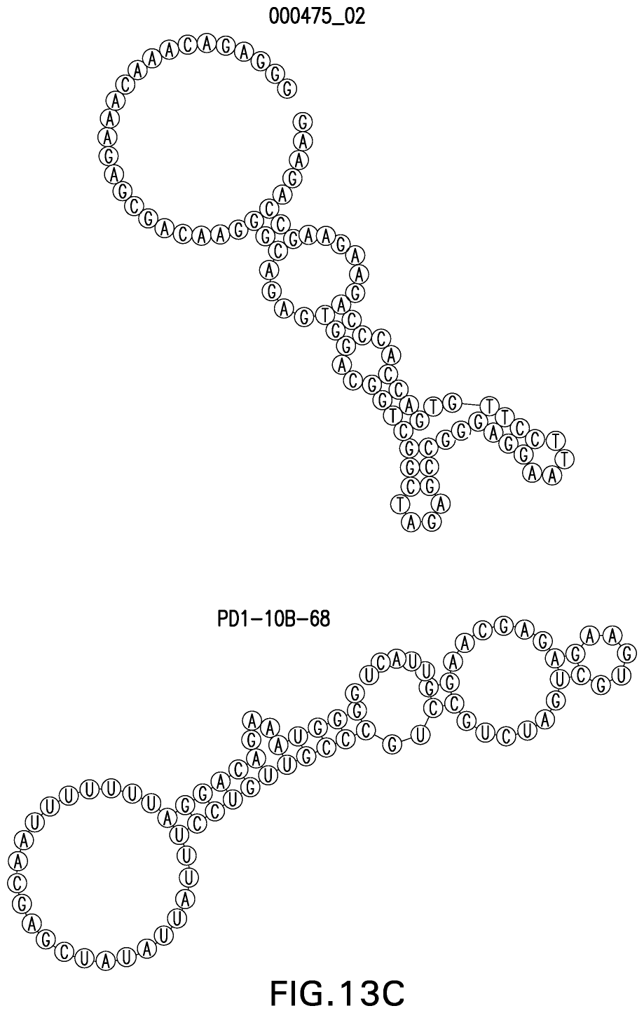

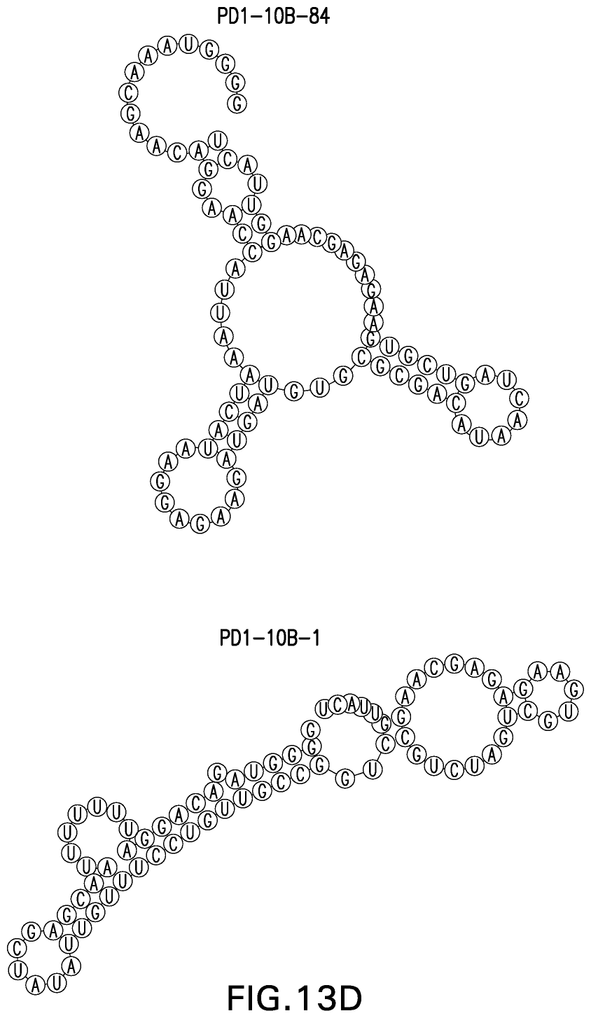

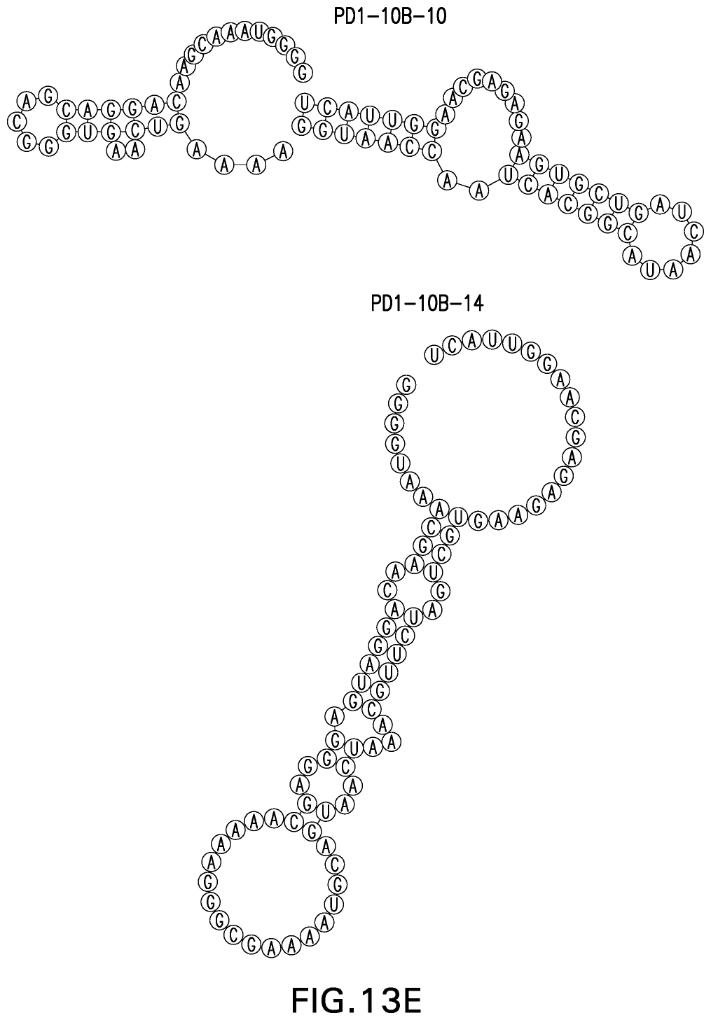

Binding structures of select aptamers are shown in FIG. 13.

CTLA-4 Aptamer Sequences
(SEQ ID NO: 99):
5′-GGGAGAGAGGAAGAGGGAUGGGCCGACGUGCCGCA-3′
2_pyridyl Modification:
(SEQ ID NO: 100):
5′-GGGAGACAAACAAAGAGCGACAAGGGCAGAAUUACAAUAG
CUAUAGUCCGGGCACCAUGCUUGUAAAUCCACCCAGAAGAAGC
CAGAAG-3′
(SEQ ID NO: 101):
5′-GGGAGACAAACAAAGAGCGACAAGGGCAGGUAGGACGCUA
GCAGACUAGAAUGUAUCUAUGCUUAGAUCCACCCAGAAGAAGC
CAGAAG-3′
(SEQ ID NO: 102):
5′-GGGAGACAAACAAAGAGCGACAAGGGCAGGCUAGUAUUAC
AAUGUCGUGGAAAAGCCGUGCGGGGUAUCCACCCAGAAGAAGC
CAGAAG-3′7
Benzyl Modification:
(SEQ ID NO: 103):
5′-GGGAGACAAACAAAGAGCGACAAGGGCAGGGAGCCAUUCU
UGAAAUUGUCAGUUUGAUUGUGCUCAGGUCACCCAGAAGAAGC
CAGAAG-3′
(SEQ ID NO: 104):
5′-GGGAGACAAACAAAGAGCGACAAGGGCAGAAAGUACAAUG
GUUGACAUAUACCGUCGGUUUAUCCUAUGCACCCAGAAGAAGC
CAGAAG-3′
(SEQ ID NO: 105):
5′-GGGAGACAAACAAAGAGCGACAAGGGCAGGUAGGCUAUCG
CUGCUUGAUCGUCUGAUCAGAGCCUAUACCACCCAGAAGAAGC
CAGAAG-3′
(SEQ ID NO: 106):
5′-GGGGUAAACGAACAGGAAACAGAUGGCCAACACAGGCGAA
GCAUAGACUAGGAACGGCUAGUCGUGAAGAGAGCAAGGUUAC
U-3′ (CTLA4-A10-6)
(SEQ ID NO: 107):
5′-GGGGUAAACGAACAGGACUUGAUGUGAAAAGGCGACGCGA
UGAGACGAAGGGCUUCUAGUCGUGAAGAGAGCAAGGUUAC
U-3′ (CTLA4-A10-38)
(SEQ ID NO: 108):
5′-GGGGUAAACGAACAGGAAGUAGACUAGACGGCGGCGAUAA
CCAGAUAACGACAUUCUCUAGUCGUGAAGAGAGCAAGGUUAC
U-3′ (CTLA4-A10-14)
(SEQ ID NO: 109):
5′-GGGGUAAACGAACAGGACCGAGUGAGACGGGUAGUGGACA
AAUGAAGUAGUGUGGUCCUAGUCGUGAAGAGAGCAAGGUUAC
U-3′ (CTLA4-A10-2)
(SEQ ID NO: 110):
5′-GGGGUAAACGAACAGGACUUUUAAUUUCACGCCGCACGAU
CCGGAAAAACGACUUGACUAGUCGUGAAGAGAGCAAGGUUAC
U-3′ (CTLA4-A10-13)

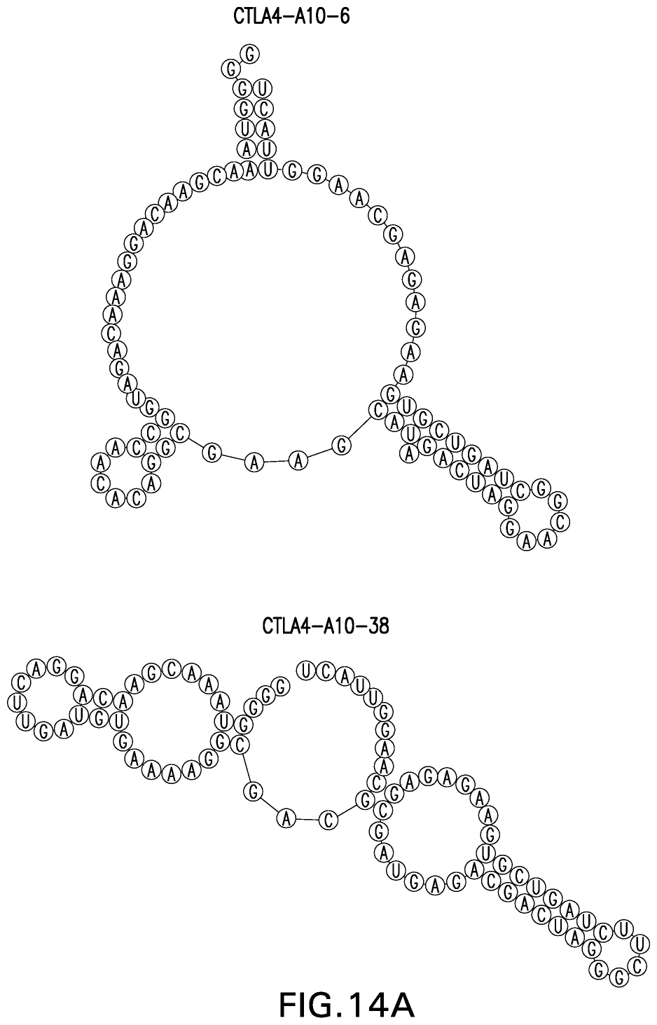

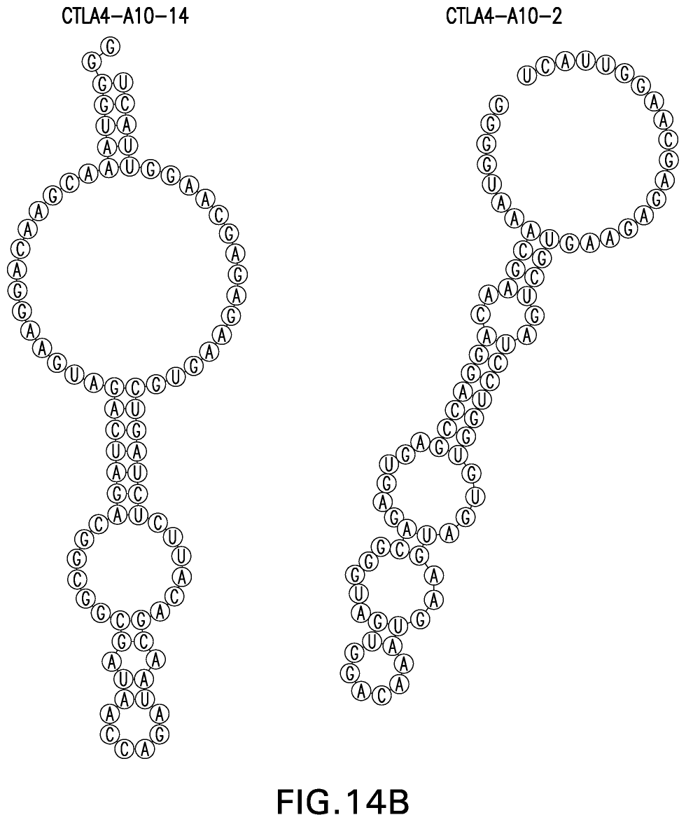

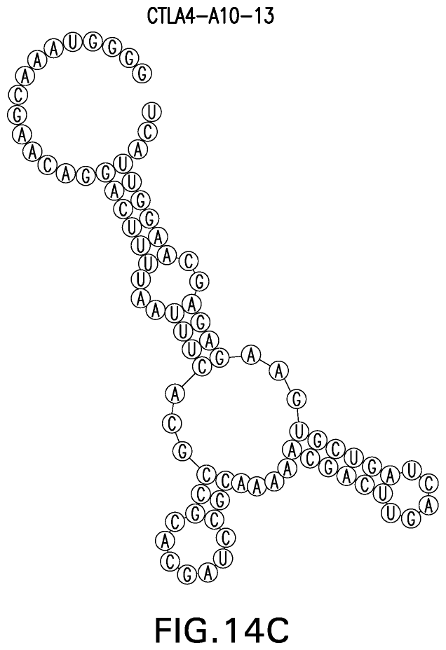

Binding structures of select aptamers are shown in FIG. 14.

siRNA Target sequences of NR4A1:
(SEQ ID NO: 111):
5′-CTGATTAATATATTTAATATA-3′
(SEQ ID NO: 112):
5′-CTCCTTCCACATGTACATAAA-3′
(SEQ ID NO: 113):
5′-CAGCATTATGGTGTCCGCACA-3′
(SEQ ID NO: 114):
5′-CAGCACCTTCATGGACGGCTA-3′
(SEQ ID NO: 115):
5′-GCACCTTCATGGACGGCTA-3′ (hNR4A1.1E2)
(SEQ ID NO: 116):
5′-GCATATGGTGTCCGCACA-3′ (hNR4A1.2E2)
(SEQ ID NO: 117):
5′-TGAAGGAAGTGTCCGAAC-3′ (hNR4A1.3E2)
(SEQ ID NO: 118):
5′-CTGCAGAACCGCATCGCCA-3′ (hNR4A1.4E2)
(SEQ ID NO: 119):
5′-TGCTGTGTGTGGGGACAAC-3′
(SEQ ID NO: 120):
5′-GGGCTGCAAGGGCTTCTTC-3′
(SEQ ID NO: 121):
5′-GCGCACAGTGCAGAAAAAC-3′
(SEQ ID NO: 122):
5′-CAGTGGCTCTGACTACT-3′
(SEQ ID NO: 123):
5′-CCACTTCTCCACACCTTGA-3′
(SEQ ID NO: 124):
5′-GGCTTGAGCTGCAGAATG-3′
(SEQ ID NO: 125):
5′-CACAGCTTGCTTGTCGATGTC-3′
(SEQ ID NO: 126):
5′-GGTCCCTGCACAGCTTGCTTGTCGA-3′
(SEQ ID NO: 127):
5′-CCGGTTCTCTGGAGGTCATCCGCAA-3′
(SEQ ID NO: 128):
5′-CAGCATTATGGTGTCCGCACATGTG-3′
siRNA Target sequences of VHL:
(SEQ ID NO: 129):
5′-AATGTTGACGGACAGCCTATT-3′
(SEQ ID NO: 130):
5′-AAGAGTACGGCCCTGAAGAAG-3′
(SEQ ID NO: 131):
5′-AAGGAGGTTTGTATAAGTAAT-3′
(SEQ ID NO: 132):
5′-CAGGAGCGCATTGCACATCAA-3′
(SEQ ID NO: 133):
5′-CCCTATTAGATACACTTCTTA-3′
(SEQ ID NO: 134):
5′-TAAGGAGGTTTGTATAAGTAA-3′
(SEQ ID NO: 135):
5′-CCTAGTCAAGCCTGAGAATTA-3′
(SEQ ID NO: 136):
5′-CTGCCAGTGTATACTCTGA-3′
(SEQ ID NO: 137):
5′-ATACACTCGGTAGCTGTGG-3′

The PCR products are processed according to the methods previously stated.

### Example 6: Building Bispecific Aptamer-siRNA: LAG-3-NR4A1-PD-1

Two RNAs are generated by in vitro transcription, with PCR products as templates.
RNA1: LAG-3 aptamer-NR4A1 antisense RNA
RNA2: PD-1 aptamer and NR4A1 sense siRNA.

The PCR products are processed as previously described.

Lag-3 Aptamer Sequence:
(SEQ ID NO: 138):
5′-GGGAGAGAGAUAUAAGGGCCUCCUGAUACCCGCUGCUA
UCUGGACCGAUCCCAUUACCAAAUUCUCUCCC-3′
(SEQ ID NO: 139):
5′-GGGGUAAACGAACAGGAAGACGGCGCAAUAAGACAGAC
UAGGACACGAUUAGAGGUACUAGUCGUGAAGAGAGCAAGGU
UACU-3′ (LAG3-A10-4)
(SEQ ID NO: 140):
5′-GGGGUAAACGAACAGGAUAAAAGAAAACAACUAGCGCG
ACGAGAGAAUAAAAUGAAACUAGUCGUGAAGAGAGCAAGGU
UACU (LAG3-A10-71)
(SEQ ID NO: 141):
5′-GGGGUAAACGAACAGGAUAAUUGUUGGGGAAAUAAAUU
GCUGGGAACGACUUAAAAGCUAGUCGUGAAGAGAGCAAGGU
UACU-3′ (LAG3-A10-79)
(SEQ ID NO: 142):
5′-GGGGUAAACGAACAGGAGUUAAUCAUGAGGUAGGUAAC
AAAAGGCAACGGCCAAUAACUAGUCGUGAAGAGAGCAAGGU
UACU (LAG3-A10-41)
(SEQ ID NO: 143):
5′-GGGGUAAACGAACAGGAUAACCAUGCAAAUAACAAGCA
AACAGAGAACUCACGCCAGCUAGUCGUGAAGAGAGCAAGGU
UACU-3′ (LAG3-A10-7)

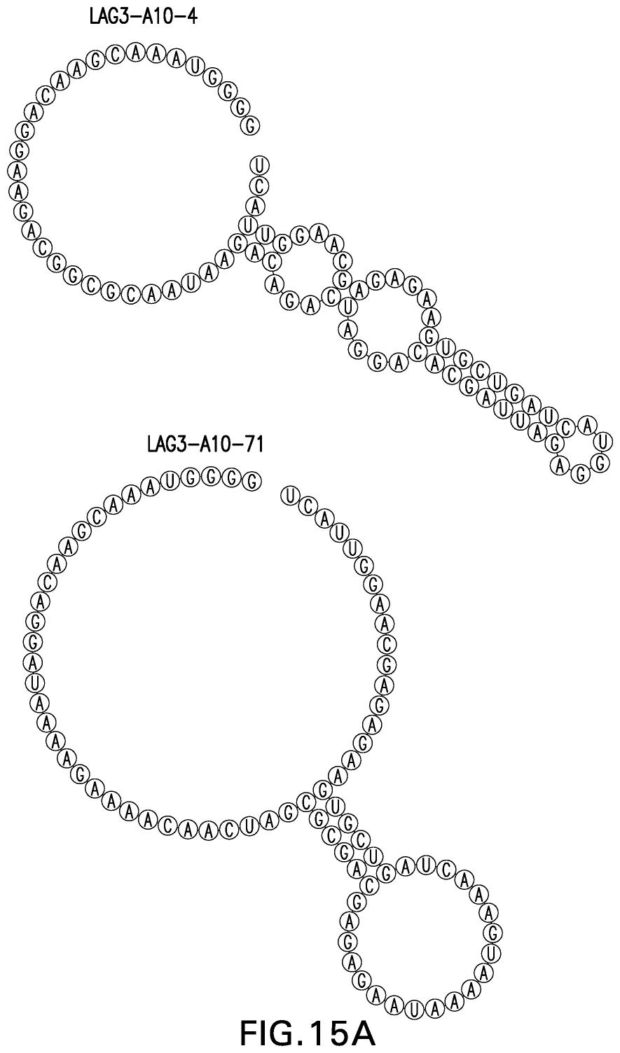

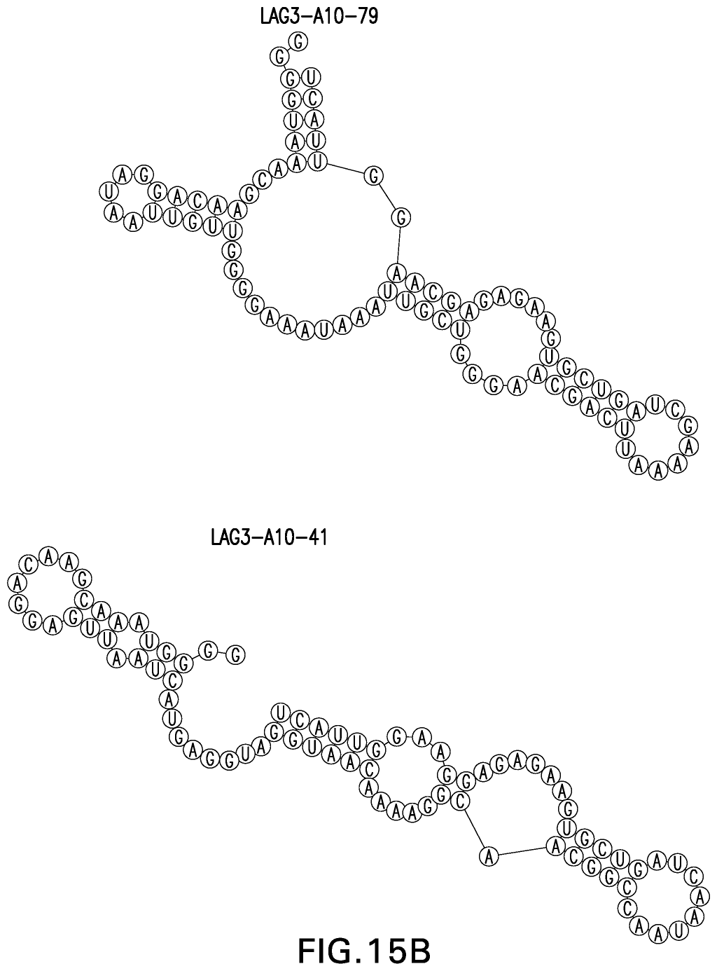

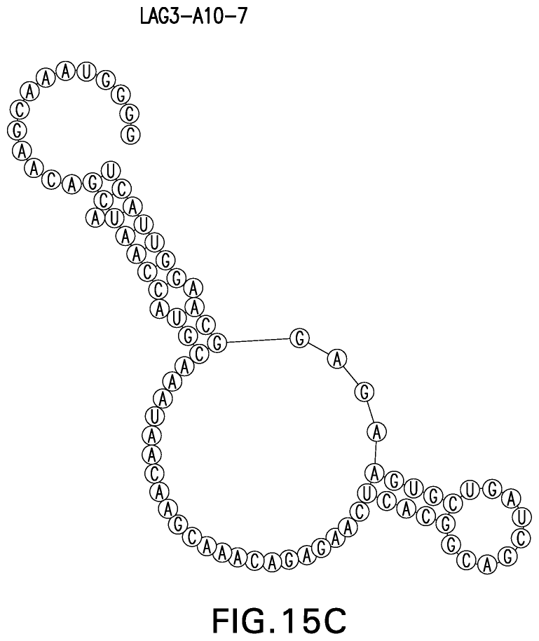

See FIG. 15 for example binding structures.

### Example 7: PD-1-NR4A1-VHL-PD-1 Construct

Three RNAs are generated by in vitro transcription, with PCR products as templates.
RNA1: PD-1 aptamer-NR4A1 antisense siRNA
RNA2: PD-1 aptamer and VHL sense siRNA and NR4A1 sense siRNA
RNA3: VHL anti-sense strand

PCR products are processed as previously discussed.

### Example 8: PD-1-CBLB-ADORA1/2-TIM3

RNA1: PD1 aptamer-CBLB antisense siRNA
RNA2: TIM3 aptamer and ADORA1/2 sense siRNA and CBLB sense siRNA
RNA3: ADORA1/2 anti-sense strand

PCR products are processed as previously discussed using sequences presented in this application.

TIM3 Aptamer Sequence:
(SEQ ID NO: 144):
5′-GGGGUAAACGAACAGGAAGGGAGUCGAUUUGAGUUGUA
AUUUGACCUAUGUUAUAAUCUAGUCGUGAAGAGAGCAAGGU
UACU-3′ (TIM3-A-4)
(SEQ ID NO: 145):
5′-GGGGUAAACGAACAGGAAUGGCUACAGUAUCGAUGCAG
UUUUCGAAUGAAGUAGAAACUAGUCGUGAAGAGAGCAAGGU
UACU-3′ (TIM3-A-8)
(SEQ ID NO: 146):
5′-GGGGUAAACGAACAGGACAGGACAGCAAGCAGUAGAAA
ACAAGCCACGAAGGGGACUCUAGUCGUGAAGAGAGCAAGGU
UACU-3′ (TIM3-A-25)
(SEQ ID NO: 147):
5′-GGGGUAAACGAACAGGAUUUUGGACUGUCUAGCCGAUG
UACUUAAGUUUAUCAUUUUCUAGUCGUGAAGAGAGCAAGGU
UACU-3′ (TIM3-A-43)
(SEQ ID NO: 148):
5′-GGGGUAAACGAACAGGAGCAGUCGCUGGCUUCAUUUUU
UUUUUUUUUUUGUGCUCAACUAGUCGUGAAGAGAGCAAGGU
UACU-3′ (TIM3-A-57)
PSMA Aptamer Sequence:
Benzyl Modified: (SEQ ID NO: 149):
5′-AGGAGACACAUGUGACAAGAGGCUAUGAUCCUGAAUGC
AUCCUUGGCAAAC-3′
4-Pyridyl Modified: (SEQ ID NO: 150):
5′-AGGAGAAUCAUGAGUUAUCUGUGUAAGGAACCAAAGCC
AUGCUUAUCAAAC-3′

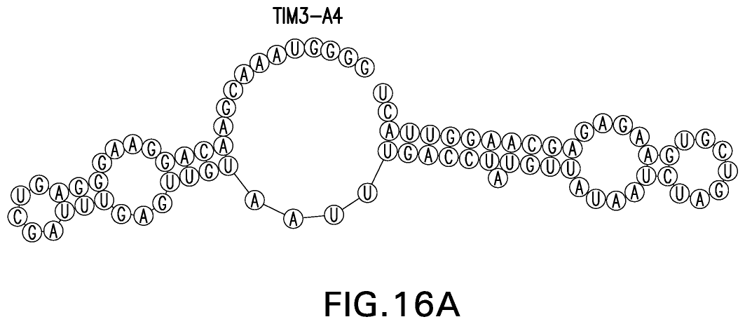

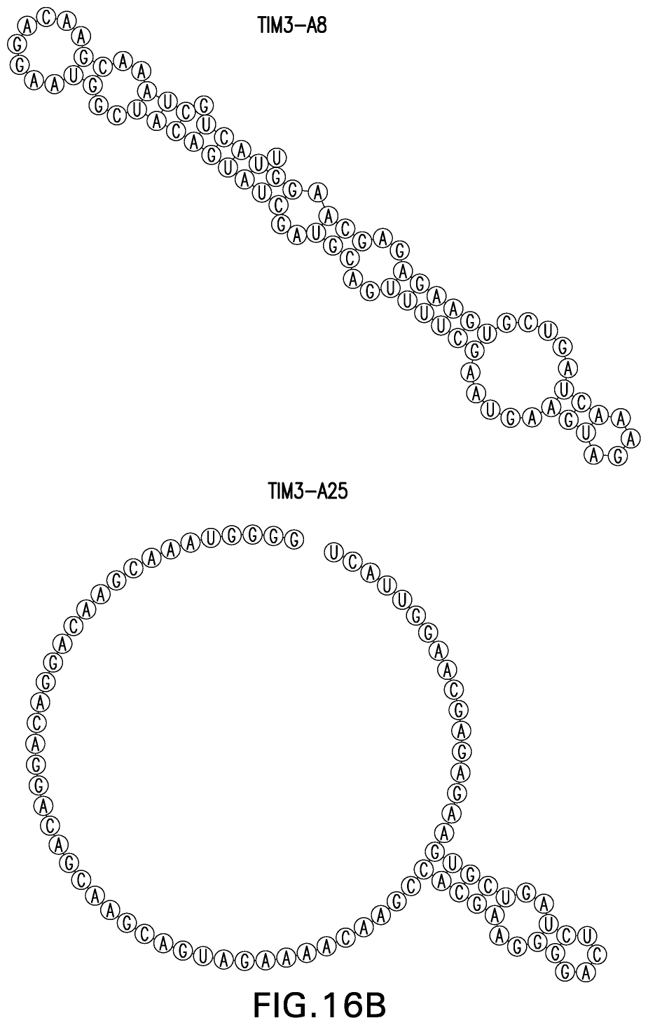

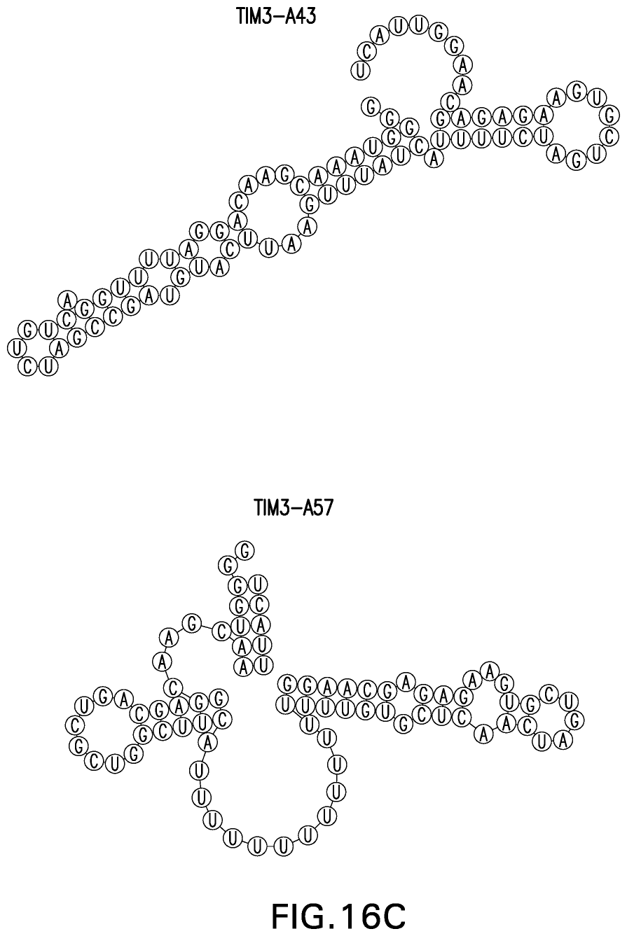

See FIG. 16 or example binding structures.

### Example 9: Reverse Chimera Linkers

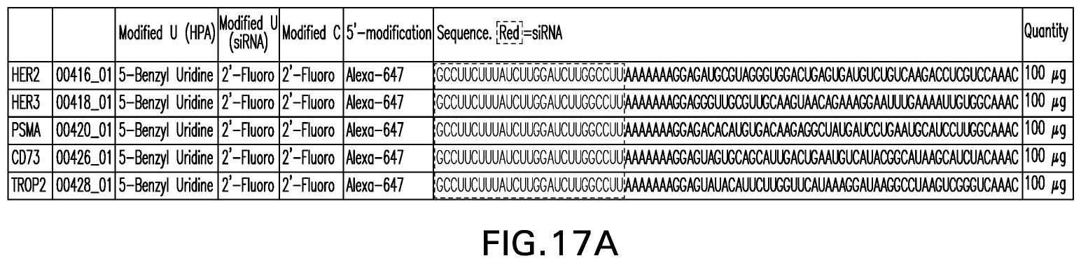

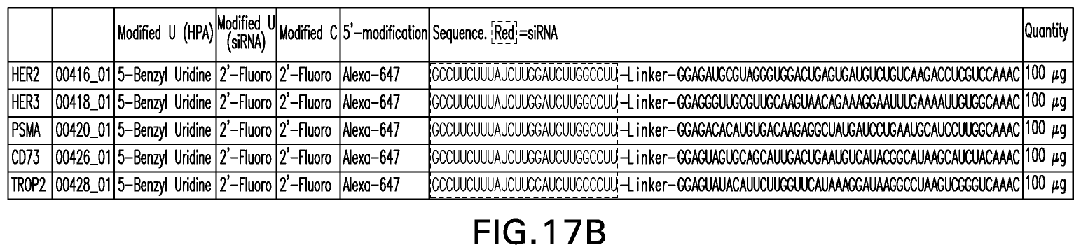

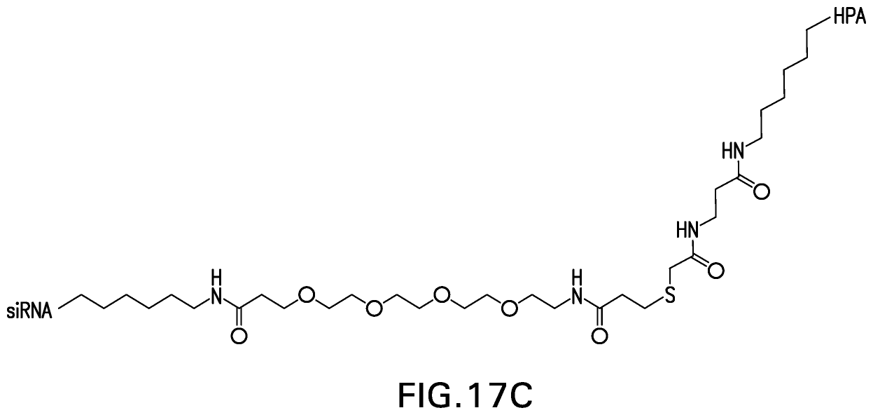

Standard linkage is 3′ end of an aptamer linked to 5′ of an siRNA. Here we provide an example of the 3′end of the siRNA linked to the 5′end of the aptamer. Linked via poly-adenosine linkage. siRNA is the guide strand (FIG. 17A). Provided is another example of a reverse chimera structure using an alternative linker shown in FIG. 17C). However, alternative linkers as previously described can be used in place here.

### Example 10: NR4A1 is Linked to CD8+ T Cell Dysfunction

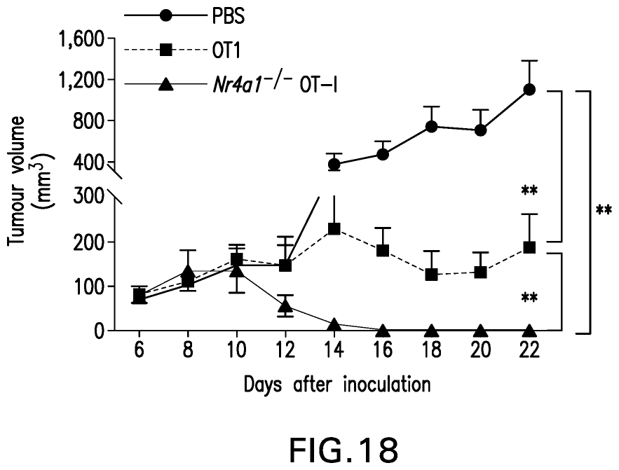

CD45.1⁺CD45.2⁺ (B6SJL xC57BL6) congenic mice were subcutaneously injected with OVA-expressing EL4 cells (E.G7 lymphoma) cells (5×10⁵ cells per mouse) in one flank. Six days later, PBS, wild-type or Nr4a1^{-/-} OT-I cells (3×10⁶ cells per mouse) were adoptively transferred into mice intravenously. Tumour sizes were monitored after adoptive transfer. To assess tumour-infiltrating donor T cells, mice were euthanized 6 days after T cell transfer. Donor-derived T cells were collected from tumour, draining lymph nodes and spleens, and subjected to flow cytometry analysis. Adoptive transfer of Nr4a1^{-/-} transgenic CD8+ T recognizing OVA₂₅₇₋₂₆₄ peptide (OT-I) cells into E.G7 tumour cell-bearing mice nearly eliminated tumours, in contrast with wild-type OT-I cells and a PBS control group. This data supports that NR4A1 is linked to CD8+ T cell dysfunction (Liu, X., et al. Nature 2019) (FIG. 18).

### Example 11: Exhausted CD8⁺ T Cells

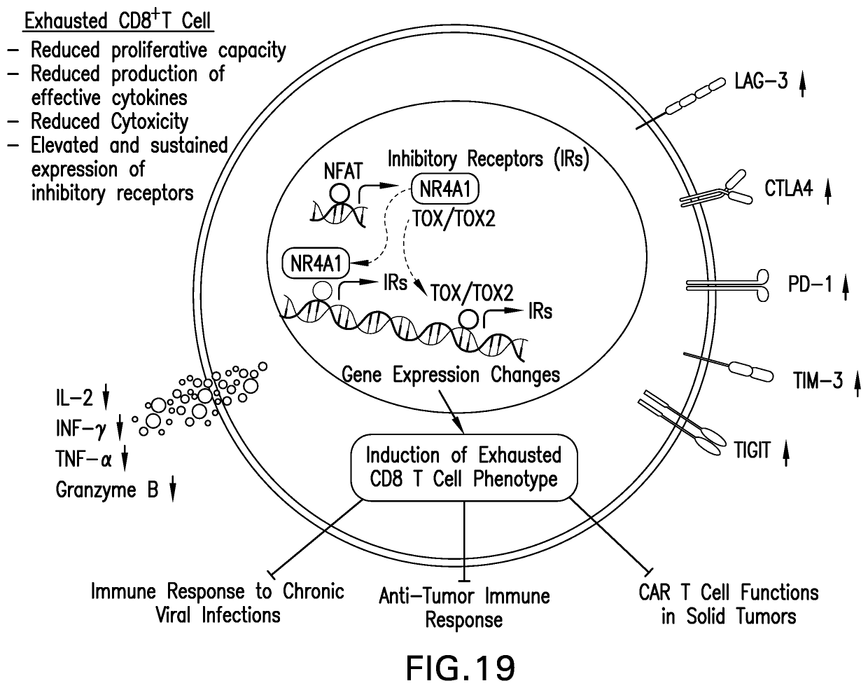

Dysfunctional, or exhausted CD8⁺ T cells arise in the settings of chronic viral infection or cancer when persistent exposure to antigen leads to prolonged T cell receptor (TCR) signaling. In the exhausted state, T cell effector functions are impaired and manifest as decreased proliferative capacity, reduced cytolytic function and effector cytokine production, and altered in gene expression and metabolism. Notably, exhausted T cells upregulate multiple inhibitory receptors that include but are not limited to these immune checkpoint proteins: PD-1, CTLA-4, TIM-3, LAG-3, TIGIT, 2B4/CD244 and others. While activated effector T cells also transiently express immune checkpoint proteins, expression level increase and are sustained on exhausted T cell subsets. Transcription factors such as TOX and NR4A1 have been described as master regulators of exhaust (FIG. 19).

### Example 12: Multimodal Immunomodulatory Chimera

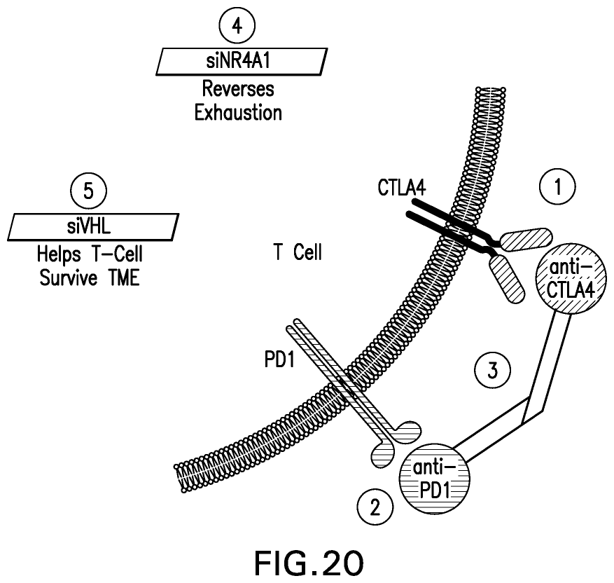

This first-in-class, bivalent aptamer-dual siRNA chimera harnesses the immune stimulatory potential of CTLA-4 and PD-1 within one RNA molecule. The results of the Phase III Checkmate 227 clinical trial in advanced non-small cell lung cancer recently demonstrated the longer duration of overall survival compared with chemotherapy in patients with NSCLC (Hellmann et al., N Engl J Med, 2019). In addition to delivering CTLA-4 and PD-1 antagonists selectively to T cells, this bivalent aptamer carries siRNA silencers that knock down expression of NR4A1, which reinvigorates exhausted T cells and VHL, which enables cells to adapt to hypoxic conditions in the TME. (FIG. 20)

In certain embodiments, the invention provides pharmaceutical compositions containing a dual targeting siRNA agent, as described herein, and a pharmaceutically acceptable carrier.

The pharmaceutical compositions featured herein are administered in dosages sufficient to inhibit expression of the target genes. In general, a suitable dose of siRNA will be in the range of 0.01 to 200.0 milligrams per kilogram body weight of the recipient per day, generally in the range of 1 to 50 mg per kilogram body weight per day.

The pharmaceutical composition may be administered once daily, or the siRNA may be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A construct comprising an aptamer that specifically binds at least one target protein and a siRNA that is processed by cellular machinery to produce one or more siRNAs and wherein the target protein is on a cell of the immune system.

2. A construct according to claim 1 wherein the cell of the immune system is a T cell.

3. A construct according to claim 2 wherein the at least one target protein is selected from PD-1, CTLA-4, TIM-3, and LAG-3.

4. A construct according to claim 2 wherein the siRNA comprises a siRNA that targets one or more of NR4A2, NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E, or STAT3.

5. A construct according to claim 3 wherein the siRNA comprises a siRNA that targets one or more of NR4A2, NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E, or STAT3.

6. A construct according to claim 4, wherein the different genes comprise NR4A1 and VHL.

7. A construct according to claim 5, wherein the different genes comprise NR4A1 and VHL.

8. A construct according to claim 3 wherein the target protein comprises PD-1.

9. A construct according to claim 3 wherein the at least one target protein comprises CTLA4.

10. A construct according to claim 2 wherein the at least one target protein comprises CTLA4 and PD-1.

11. A construct according to claim 10 wherein the different genes comprise NR4A1 and VHL.

12. A construct according to claim 4 further comprising unpaired linkers comprising two to six adenines between each aptamer and siRNA and between each siRNA.

13. A construct according to claim 5 further comprising unpaired linkers comprising two to six adenines between each aptamer and siRNA and between each siRNA.

14. The construct of claim 12 wherein the linker is a poly-adenosine linker.

15. The construct of claim 13 wherein the linker is a poly-adenosine linker.

16. A construct comprising:
a. first and second ends, wherein the first and second ends comprise an aptamer that specifically binds a target protein selected from PD-1, CTLA-4, TIM-3 and LAG-3;
b. an siRNA construct between the first and second ends, wherein the siRNA construct comprises a siRNA that targets one or more of NR4A2, NR4A1, NR4A2, NR4A3, VHL, ADORA2A, ADORA2B, PTPN2, CBLB, TOX, TOX2, YY1, BATF, PDCD1, TIGIT, LAG3, HAVCR2, CTLA4, NT5E and STAT3; and
c. unpaired linkers comprising two to four adenines between each aptamer and
siRNA and between each siRNA.
